(19) United States
(12) Patent Application Publication
(10) Pub. No.: US 2011/0002981 A1
(43) Pub. Date: Jan. 6, 2011

(54) MCPIP Protection Against Cardiac Dysfunction

(76) Inventors: Pappachan E. Kolattukudy, Orlando, FL (US); Jianli Niu, Orlando, FL (US)

Correspondence Address: Timothy H. Van Dyke, 390 No. Orange Avenue, Suite 2500, Orlando, FL, 32801, US

(21) Appl. No.: 12/859,558

(22) Filed: Aug. 19, 2010

Related U.S. Application Data
(63) Continuation-in-part of application No. 12/539,907, filed on Aug. 12, 2009.
Application No. 12/539,907 is a continuation of application No. 11/643,057, filed on Dec. 20, 2006, now abandoned.

(51) Int. Cl. A61K 9/127; A61K 38/46; A61K 31/7088; A61K 35/12; A61P 29/00

(52) U.S. Cl. 424/450; 424/94.6; 514/44 R; 424/93.7

(57) ABSTRACT

Disclosed herein are methods and compositions of treating a patient at risk of experiencing sepsis induced cardiac dysfunction. In exemplary examples, the method involves elevating MCPIP levels in a patient in need. Elevating MCPIP levels may involve direct administration (e.g. delivery of protein) or indirect administration (e.g. delivery vehicle capable of increasing expression of MCPIP).

### RELATED APPLICATIONS

This application is a continuation-in-part of U.S. Ser. No. 12/539,907 filed Aug. 12, 2009, which is a continuation of U.S. Ser. No. 11/643,057 filed Dec. 20, 2006, no abandoned which are incorporated herein in their entirety.

### BACKGROUND

Sepsis remains a leading and growing cause of death in hospitalized patients with an incidence of 400,000-750,000 per year and is lethal in 20-30% of patients in the United States (1). Endotoxin (lipopolysaccharides, LPS) is believed to be responsible for triggering systemic inflammatory responses leading to septic shock and multiple organ dysfunction with high mortality and morbidity (2). The development of myocardial dysfunction occurs in 40-50% of patients with prolonged septic shock, and its occurrence increases mortality in patients with sepsis from 20% to 70-90% (2, 3). Although the precise mechanisms responsible for myocardial dysfunction in the setting of sepsis remain undefined, increasing evidence suggests that LPS provokes host cells to produce a large amount of inflammatory cytokines, including tumor necrosis factor (TNF)-α, interleukin (IL)-1β, IL-6, and the cytokine-inducible nitric oxide synthase (iNOS), acting directly or indirectly to cause cardiac myocyte injury leading to myocardial dysfunction (4, 5). These cytokines were originally thought to be produced by circulating lymphocytes such as macrophages stimulated by LPS. However, recent evidence that a functional innate immune signaling system exists in the heart and suggests a direct role of cardiac myocytes in the release of these inflammatory mediators and reactive oxygen/nitrogen species (ROS/RNS) (6). The activity of these inflammatory mediators in the heart explains, to a considerable extent, the functional depression of myocardium in sepsis, and activation of cardiac nuclear factor-kappaB (NF-κB) signaling by LPS is thought to be a key molecular event involved in this process (7, 8). NF-κB is present in the cytoplasm forming an inactive complex consisting of p50, p65 and IKBα subunits under the noninflammatory state. LPS-induced NF-κB activation is triggered by the interaction between the toll-like receptor 4 (TLR-4) and myeloid differentiation protein 88 (MyD88), followed by stimulation of intracellular signal cascades that involve the recruitment of downstream adaptor molecules and kinases, resulting in activation of inhibitor kappa B kinase (IKK) complex and subsequent release of NF-κB for nuclear translocation and binding to specific sequences in the DNA, thus causing transcriptional activation of a broad spectrum of inflammatory genes (9, 10). Inhibition of NF-κB activation has been shown to restore systemic hypotension and attenuate septic myocardial dysfunction in animal models of sepsis (8, 11-13) and improve survival in patients with severe sepsis (14, 15). NF-κB activation in cardiomyocytes has also been linked to myocardial hypertrophy, inflammation and the development of heart failure (16-18). Thus, the prolonged cardiac NF-κB activation is critical to the development of myocardial dysfunction in various pathological states.

Recently, using a gene array approach we discovered that signaling initiated monocyte chemotactic protein-1 (MCP-1) binding to its receptor CCR2 in human peripheral blood monocytes triggered the induction of a novel ZCCCH-zinc finger protein named MCPIP (MCP-1-induced protein) (19). We previously demonstrated that MCPIP was significantly induced in the infiltrating inflammatory cells and the major cell types in the myocardium in response to inflammatory insults induced by cardiac-specific expression of MCP-1, and observed an elevated level of MCPIP expression in the MCP-1 transgenic hearts that developed ischemic cardiomyopathy [19].

### SUMMARY

Whether MCPIP is involved in the development of cardiac pathologies or it represents an effort to protect heart from inflammatory injury remains unknown. Recently, the inventors found that MCPIP inhibited LPS-stimulated production of inflammatory mediators such as TNF-α, IL-1β, IL-6 and iNOS in macrophages (20). The inhibitory effects of MCPIP on production of inflammatory cytokines in macrophages and other cell types are mainly mediated by interference with the NF-κB signaling pathway (20, 21). In addition, it has been reported that MCPIP can negatively regulate IL-6 and IL-1β production by its RNase activities (22, 23). The inventors have discovered that MCPIP expression mitigates septic cardiac dysfunction by modulating myocardial inflammatory state. The inventors developed transgenic mice expressing MCPIP in the myocardium under the control of the α-myosin heavy chain (MHC) promoter. The results presented herein demonstrate that over-expression of MCPIP in the myocardium leads to the reduction of pro-inflammatory cytokines, inhibition of iNOS expression and nitrosative stress, and dramatically improved myocardial function in LPS model of sepsis by blocking cardiac NF-κB signaling pathway through IKK inactivation. This constitutes the first in vivo evidence for the protective role of MCPIP against septic cardiac dysfunction.

According to one embodiment, the invention relates to treating or preventing septic cardiac dysfunction by elevating MCPIP levels in a patient in need. Included in the present description are several methods of elevating MCPIP for treatment or prevention of septic cardiac dysfunction are taught herein. Such examples are not intended to be limiting but are provided as embodiments that may be implemented to elevate MCPIP in the particular patient population either exhibiting or at risk of developing septic cardiac dysfunction.

### BRIEF DESCRIPTION OF THE DRAWINGS

### Figure Legends

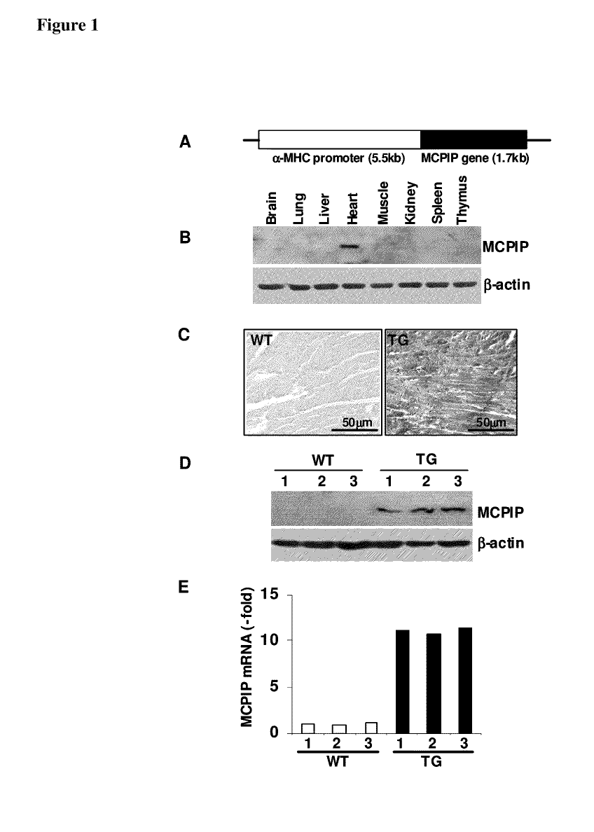

FIG. 1. Generation of MCPIP TG mice. (A) Schematic representation of the transgene construct used to generate MCPIP TG mice. MCPIP expression is controlled by the cardiac-specific promoter α-myosin heavy chain (α-MHC). (B) Representative immunoblot analysis of MCPIP protein from different tissue of TG mice. (C) Detection of MCPIP protein by immunoperoxidase reactivity is seen in MCPIP TG cardiac tissue, but not in cardiac tissue from nontransgenic mice. (D, E) Immunoblot and real-time PCR analysis of MCPIP expression in the heart tissue from individuals of TG and age-, sex-matched WT mice showed stable inheritance of the transgene in the myocardium.

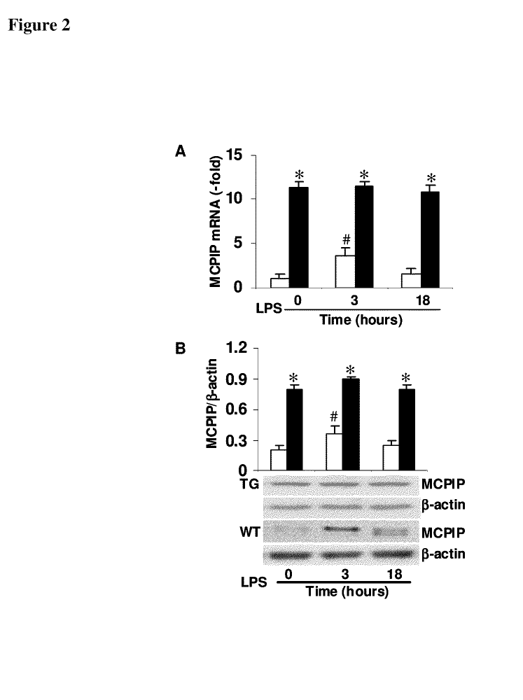

FIG. 2. Effect of LPS exposure on MCPIP expression in the myocardium. Real-time PCR (A) and Immunoblot (B) analysis of MCPIP expression in the myocardium from both TG and age-, sex-matched WT mice after LPS exposure at the time points indicated. Values presented are means±SEM. n=3 per group at each time point. *P<0.01 compared with WT groups; ^{#}P<0.05 compared with other time points in WT mice.

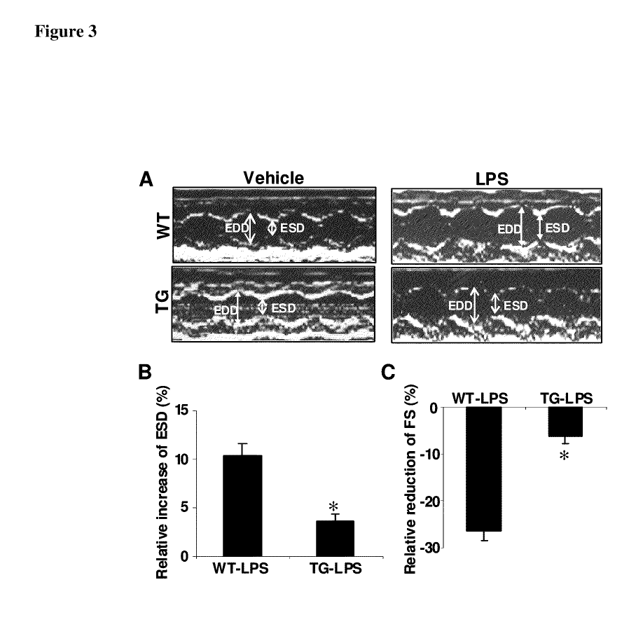

FIG. 3. Cardiac-specific expression of MCPIP attenuates LPS-induced cardiac dysfunction. (A) Representative M-mode echocardiograms obtained from WT and MCPIP TG mice before and after 18 hr of LPS exposure. EDD and ESD indicate left ventricular end-diastolic diameter and end-systolic diameter, respectively. (B) The chart represents the percentage increase in left, ventricular ESD after 18 hr of LPS exposure as compared with its own normal baseline data. Compared with LPS-treated WT mice, MCPIP TG mice showed much less increase in left ventricular ESD after LPS challenge. Values presented are means±SEM. n=5 per group, *P<0.05 compared with LPS-treated WT mice. (C) The bar chart represents the percentage reduction in left ventricular fractional shortening (FS) after 18 hr of LPS exposure as compared with its own normal baseline data. Compared with LPS-treated WT mice, MCPIP TG mice showed only a slight decrease in FS % after LPS challenge. Values presented are means±SEM. n=5 per group, *P<0.05 compared with LPS-treated WT mice.

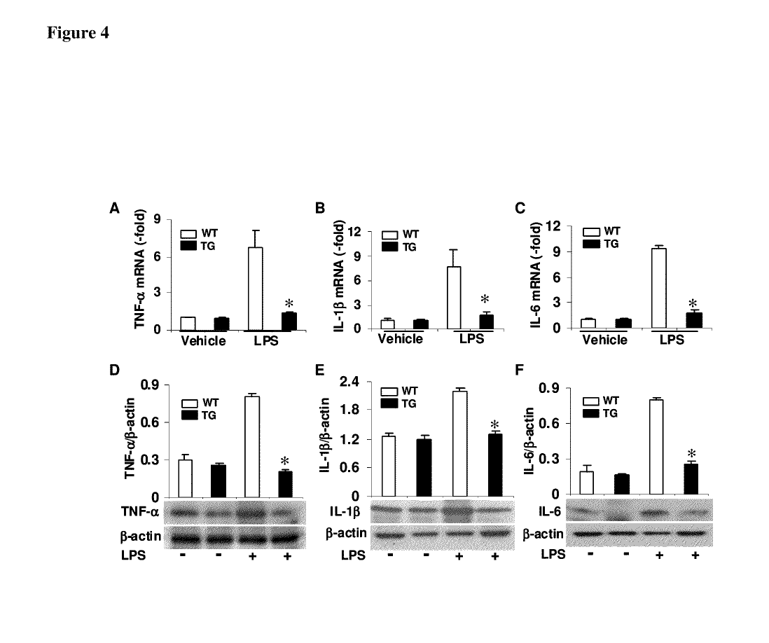

FIG. 4. Cardiac-specific expression of MCPIP inhibits LPS-induced inflammatory cytokine production in the myocardium. (A-C) Expression of TNF-α, IL-1β, and IL-6 mRNA levels in the myocardium obtained from vehicle- and LPS-treated WT and MCPIP TG mice were assayed by quantitative real-time PCR. Compared with vehicle-treated WT and TG mice, LPS-treated WT mice showed a marked increase in cardiac levels of TNF-α, IL-1β, and IL-6 mRNA, which was significantly reduced in LPS-treated MCPIP TG mice. Values presented are means±SEM. n=5 per group, *P<0.05 compared with other three groups. (D-F) Immunoblot analysis of TNF-α, IL-1β, and IL-6 protein levels in the myocardium obtained from vehicle- and LPS-treated WT and TG mice. The bar graph represents the densitometric quantification of immunoblotting bands. Compared with vehicle-treated WT and TG mice, LPS-treated WT mice showed a marked increase in TNF-α, IL-1β, and IL-6 protein levels in the myocardium, which was significantly reduced in LPS-treated MCPIP TG mice. Values presented are means±SEM. n=5 per group, *P<0.05 compared with other three groups.

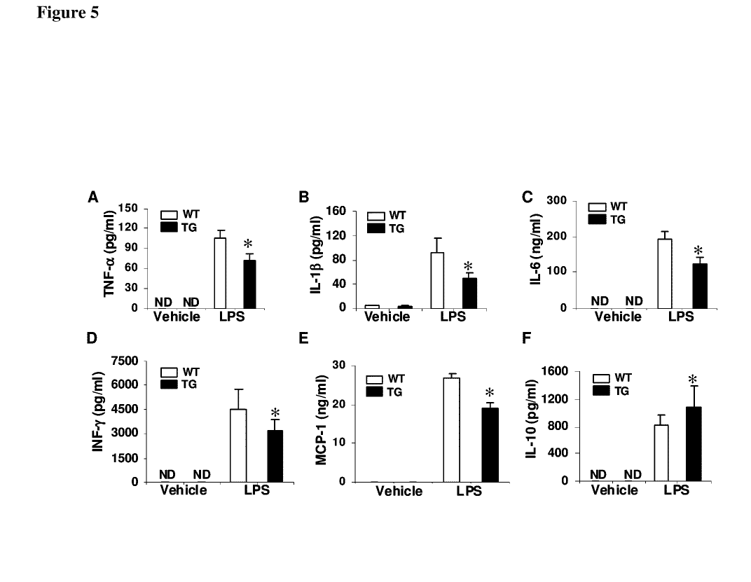

FIG. 5. Effect of cardiac-specific expression of MCPIP on serum pro-/anti-inflammatory cytokine production induced by LPS. Serum of animals divided in the four experimental groups described in Materials and methods were collected at 18 hr after LPS exposure, and levels of TNF-α, IL-1β, IL-6, IFN-γ, MCP-1, and IL-10 were determined with the Bio-Plex system. Levels of TNF-α, IL-1β, IL-6, INF-γ, and MCP-1 in the serum from LPS-treated MCPIP TG mice was significantly less, but levels of IL-10 was significant higher that that from LPS-treated WT mice. Values presented are means±SEM. n=5 per group, *P<0.05 compared with LPS-treated WT mice.

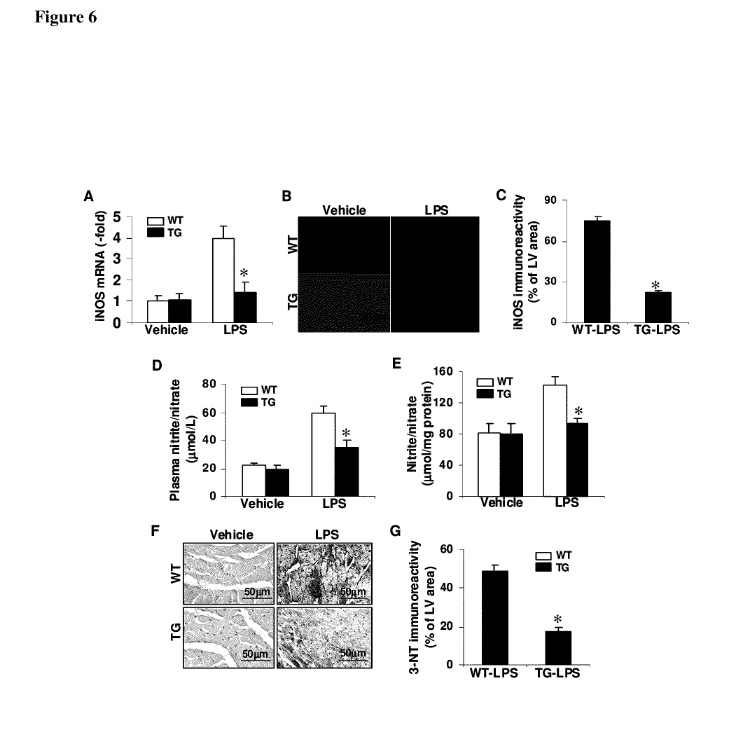

FIG. 6. Cardiac-specific expression of MCPIP attenuates LPS-induced iNOS expression and peroxynitrite formation in the myocardium. (A) iNOS mRNA levels in the myocardium of vehicle- and LPS-treated WT and MCPIP TG mice was assayed by quantitative real-time PCR. Compared with vehicle-treated WT and TG mice, LPS-treated WT mice showed a marked increase in cardiac iNOS mRNA levels, which significantly reduced in the myocardium of LPS-treated MCPIP TG mice. Values presented are means±SEM. n=5 per group, *P<0.05 compared with LPS-treated WT mice. (B) Representative photomicrographs of immunohistochemical staining revealing a strong immunoreactivity for iNOS in cardiac sections from LPS-treated WT mice as compared with very little immunoreactivity in the myocardium of LPS-treated MCPIP TG mice. Immunoreactivity was visualized with diaminobenzidine (brown). (C) Histograms showing a significant decrease of the immunoreactivity for iNOS in the myocardium of LPS-treated MCPIP TG mice compared with LPS-treated WT mice *P<0.05, n=3 per group. Plasma (D) and cardiac (E) levels of nitrite/nitrate in vehicle- and LPS-treated WT and MCPIP TG mice was assayed using commercial assay kits. Compared with vehicle-treated WT and TG mice, LPS-treated WT mice showed a marked increase in both plasma and cardiac levels of nitrite/nitrate, which significantly reduced in LPS-treated MCPIP TG mice. Values presented are means±SEM. n=5 per group, *P<0.05 compared with LPS-treated WT mice. (F) Representative photomicrographs of immunohistochemical staining showing a strong immunoreactivity for 3-nitrotyrosine (3-NT) in LPS-treated WT heart section and significantly reduced in LPS-treated MCPIP TG heart section. Immunoreactivity was visualized with diaminobenzidine (brown). (G) Histograms showing a significant decrease of the immunoreactivity for 3-NT in the myocardium of LPS-treated MCPIP TG mice compared with LPS-treated WT mice. *P<0.05, n=3 per group.

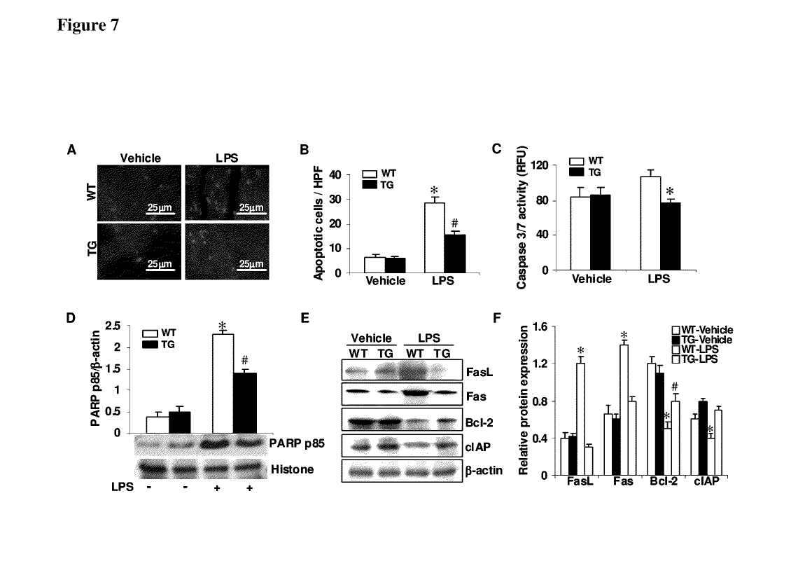

FIG. 7. Cardiac-targeted expression of MCPIP inhibits LPS-induced apoptosis and modulates apoptosis-related gene expression in the myocardium. (A) Representative photomicrographs of TUNEL-stained sections obtained from vehicle- and LPS-treated WT and MCPIP TG mice. TMR read fluorescence labeling indicates TUNEL-positive cells. (B) Histogram showing a marked decrease of TUNEL-positive cells in the myocardium of LPS-treated MCPIP TG mice compared with LPS-treated WT mice. Values presented are means±SEM. n=3 per group, *P<0.05 compared with other three groups. ^{#}P<0.05 compared with vehicle-treated WT and TG mice. (C) Caspase-3/7 activity in heart homogenates was determined quantitatively using APO-One caspase-3/7 assay. Values presented are means±SEM. n=3 per group. *P<0.05 compared with LPS-treated WT mice. (D) Detection of p85 PARP fragment in nucleus extracts from vehicle- and LPS-treated WT and MCPIP TG heart was assayed by immunoblot. The bar graph represents the densitometric quantification of immunoblotting bands. Values presented are means±SEM. n=3 per group, *P<0.05 versus other three groups; ^{#}P<0.05 versus vehicle-treated WT and TG mice. (E) Immunoblot analysis of pro-apoptotic proteins, Fas and FasL, and anti-apoptotic proteins, Bcl-2 and cIAP, in the myocardium from four groups of animals. The bar graph represents the densitometric quantification of immunoblotting bands (F). Values presented are means±SEM. n=3 per group, *P<0.05 versus other three groups; ^{#}P<0.05 versus vehicle-treated WT and TG mice.

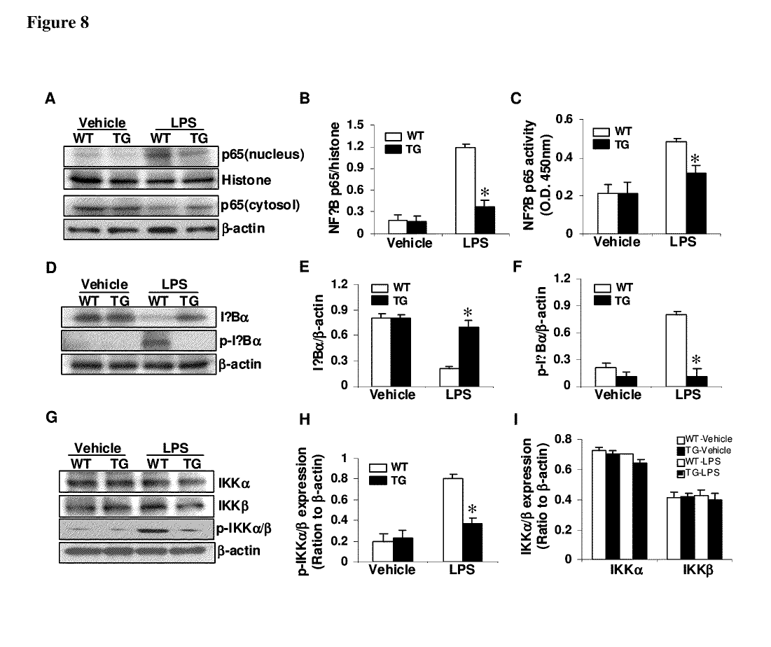

FIG. 8. Cardiac-targeted expression of MCPIP blocks LPS-induced NF-κB activation in the myocardium by inhibition of IKK activation. (A, B) Cardiac-targeted expression of MCPIP inhibits LPS-induced p65 nuclear translocation. Nuclear and cytosolic extracts were isolated from vehicle- and LPS-treated WT and MCPIP TG heart and the NF-κB p65 protein levels were assayed by immunoblot (A). Densitometry measurements of the nuclear p65 protein are normalized to histone and shown in the bar graph (B). Values presented are means±SEM. n=3 per group, *P<0.05 versus other three groups. (C) NF-κB p65 DNA binding activity as determined by ELISA was significantly higher in LPS-treated WT heart and was attenuated in LPS-treated MCPIP TG heart. Values presented are means±SEM. n=3 per group, *P<0.05 versus LPS-treated WT mice. (D-F) Cardiac-targeted expression of MCPIP blocks LPS-induced degradation and phosphorylation of IκBα. Cytosolic extracts were isolated from vehicle- and LPS-treated WT and MCPIP TG heart and IκBααdegradation and phosphorylation were assayed by immunoblot (C). Densitometry measurements of the cytosolic IκBα and phosphorylated IκBα (p-IκBα) proteins are normalized to β-actin and shown in the bar graphs (E, F). Values presented are means±SEM. n=3 per group, *P<0.05 versus LPS-treated WT mice. (G-I) Cardiac-targeted expression of MCPIP blocks LPS-induced IKK phosphorylation. Cytosolic extracts were isolated from vehicle- and LPS-treated WT and MCPIP TG heart and IKKα/β phosphorylation in cytosolic extracts of myocardium was assayed by immunoblot (G). Densitometry measurements of the phosphorylated IKKα/β (p-IKKα/β) and IKKα/β proteins in the cytosolic extracts are normalized to β-actin and shown in the bar graphs (H, I). Values presented are means±SEM. n=3 per group, *P<0.05 versus LPS-treated WT mice.

### DETAILED DESCRIPTION

The inventors have previously identified the novel transcription factor designated as MCPIP (MCP-1-induced protein). MCPIP was initially isolated from human monocytes after stimulation with MCP-1. The nucleotide (SEQ ID NO: 1) and amino acid (SEQ ID NO: 2) sequences of isolated human MCPIP were deposited with GenBank under accession number AY920403 and the nucleotide (SEQ ID NO: 3) and amino acid (SEQ ID NO: 4) sequences of isolated mouse MCPIP were deposited with GenBank under accession number AY920404. The inventors have continued to study the biological relevance of these genes/proteins, and to develop new therapies based on this research. The invention is based on the inventors discovery that maintaining or elevating MCPIP levels can protect a subject against cardiac dysfunction associated with sepsis.

According to one embodiment, the invention pertains to a method of treating or preventing cardiac septic dysfunction by elevating MCPIP levels in a patient in need. MCPIP may be elevated by direct administration of a composition. The composition may comprise a therapeutically effective amount of the MCPIP protein. The MCPIP protein composition may comprise a pharmaceutically acceptable excipient. Alternatively, MCPIP may be elevated by administering a composition comprising a delivery vehicle that results in the in vivo expression of MCPIP. In yet another embodiment, MCPIP is elevated by inducing expression of the endogenous gene. In a specific embodiment, MCPIP expression is upregulated in targeted tissue such as the heart or vasculature.

As used herein, therapeutically effective amount refers to an amount sufficient to elicit the desired biological response. In the present invention the desired biological response can be an overall improvement in the condition being treated. The overall improvement can be associated with improvement in individual symptoms. For example, a desired biological response can include improvement (complete or partial reduction) of at least one symptom associated with the following:

1) Systemic inflammatory response syndrome (SIRS): The systemic inflammatory response to a wide variety of severe clinical insults manifests by 2 or more of the following conditions:
Temperature greater than 38° C. or less than 36° C.
Heart rate greater than 90 beats per minute (bpm)
Respiratory rate greater than 20 breaths per minute or PaCO₂ less than 32 mm Hg
White blood cell count greater than 12,000/μL, less than 4000/μL, or 10% immature (band) forms

2) Sepsis: This is a systemic inflammatory response to a documented infection. The manifestations of sepsis are the same as those previously defined for SIRS. The clinical features include 2 or more of the following conditions as a result of a documented infection:
Rectal temperature greater than 38° C. or less than 36° C.
Tachycardia (>90 bpm)
Tachypnea (>20 breaths per min)

With sepsis, at least 1 of the following manifestations of inadequate organ function/perfusion also must be included:
Alteration in mental state
Hypoxemia (PaO₂ <72 mm Hg at FiO₂ [fraction of inspired oxygen] 0.21; overt pulmonary disease not the direct cause of hypoxemia)
Elevated plasma lactate level
Oliguria (urine output <30 mL or 0.5 mL/kg for at least 1 h)

3) Severe sepsis: This is sepsis and SIRS associated with organ dysfunction, hypoperfusion, or hypotension. Hypoperfusion and perfusion abnormalities may include, but are not limited to, lactic acidosis, oliguria, or an acute alteration in mental status. The systemic response to infection is manifested by 2 or more of the following conditions:
Temperature greater than 38° C. or less than 36° C.
Heart rate greater than 90 bpm
Respiratory rate greater than 20 breaths per minute or PaCO₂ less than 32 mm Hg
White blood cell count greater than 12,000/μL, less than 4000/μL, or 10% immature (band) forms

4) Sepsis-induced cardiac dysfunction
systolic blood pressure <90 mm Hg or a reduction of >40 mm Hg from baseline: This may develop despite adequate fluid resuscitation, along with the presence of perfusion abnormalities that may include lactic acidosis, oliguria, or an acute alteration in mental state.
decreased left ventricular ejection fraction (LVEF) and an acutely dilated left ventricle, as evidenced by an increased left ventricular end-diastolic volume index (LVEDVI)
low or decreasing cardiac index as calculated by the following formula.

$$CI = \frac{CO}{BSA} = \frac{SV * HR}{BSA}$$

where

CI=Cardiac index

BSA=Body surface area

SV=Stroke volume

HR=Heart rate

CO=Cardiac output

An index below 2.6 L/min per square meter is considered below normal. In particular, and index below 1.8 L/min/sq. meter is indicative of cardiogenic shock.
decreased contractility
impaired ventricular response to fluid therapy
lactic acidosis
oliguria

Subject, as used herein, refers to animals such as mammals, including, but not limited to, primates (e.g., humans), cows, sheep, goats, horses, pigs, dogs, cats, rabbits, guinea pigs, rats, mice or other bovine, ovine, equine, canine, feline, rodent or murine species. In one embodiment, the subject is a human.

In typical embodiment, a patient in need is a subject exhibiting two or more symptoms associated with sepsis-induced cardiac dysfunction.

### Modes of Administration

The compounds for use in the method of the invention can be formulated for oral, transdermal, sublingual, buccal, parenteral, rectal, intranasal, intrabronchial or intrapulmonary administration. Oral administration is preferred. For oral administration the compounds can be of the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY film coating systems available from Colorcon, West Point, Pa. (e.g., OPADRY, OY Type, OY-C Type, Organic Enteric OY-P Type, Aqueous Enteric OY-A Type, OY-PM Type and OPADRY® White, 32K18400).

In a particular embodiment, the oral form is a tablet containing MCPIP and a pharmaceutically acceptable excipient, such as, but not limited to, mannitol, corn starch, microcrystalline cellulose, colloidal silicon dioxide, polyvinyl pyrrolidone, talc, magnesium stearate, and the like which are optionally coated with an OPADRY film coating.

Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

For buccal administration, the compounds for use in the method of the invention can be in the form of tablets or lozenges formulated in a conventional manner.

For parenteral administration, the compounds for use in the method of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

For rectal administration, the compounds for use in the method of the invention can be in the form of suppositories or enemas.

For sublingual administration, tablets can be formulated in conventional manner.

For intranasal, intrabronchial or intrapulmonary administration, conventional formulations can be employed.

Further, the compounds (e.g. protein or delivery vehicle) for use in the method of the invention can be formulated in a sustained release preparation. For example, the compounds can be formulated with a suitable polymer or hydrophobic material which provides sustained and/or controlled release properties to the active agent compound. As such, the compounds for use the method of the invention can be administered in the form of microparticles for example, by injection or in the form of wafers or discs by implantation.

In accordance with the method of the invention, an expression vector is a viral or a non-viral expression vector. Viral expression vectors which may be used advantageously in the method of the invention include, but are not limited to, an adeno associated virus (AAV) vector, a lentivirus vector, an adenovirus vector, and a herpes simplex virus (HSV) vector.

### Delivery Vehicles

1) Viral Vectors

In a preferred embodiment, the compositions of the invention can be tailored to include a nucleic acid sequence (e.g., SEQ ID NO: 1, derivatives, fragments and variants thereof) in an expression vector. Viral vectors for use in the invention are those that exhibit low toxicity to a host cell. Viral vector methods and protocols that may be used in the invention are reviewed in Kay et al. Nature Medicine 7:33-40, 2001. The use of specific vectors, including those based on adenoviruses, adeno-associated viruses, herpes viruses, and retroviruses are described in more detail below.

The use of recombinant adenoviruses as vectors is discussed in W. C. Russell, Journal of General Virology 81:2573-2604, 2000; and Bramson et al., Curr. Opin. Biotechnol. 6:590-595, 1995. Adenovirus vectors are preferred for use in the invention because they (1) are capable of highly efficient gene expression in target cells and (2) can accommodate a relatively large amount of heterologous (non-viral) DNA. A preferred form of recombinant adenovirus is a “gutless”, “high-capacity”, or “helper-dependent” adenovirus vector. Such a vector features, for example, (1) the deletion of all or most viral-coding sequences (those sequences encoding viral proteins), (2) the viral inverted terminal repeats (ITRs) which are sequences required for viral DNA replication, (3) up to 28-32 kb of “exogenous” or “heterologous” sequences (e.g., sequences encoding an ammonia producing enzyme), and (4) the viral DNA packaging sequence which is required for packaging of the viral genomes into infectious capsids. For specifically targeting liver, preferred variants of such recombinant adenoviral vectors contain tissue-specific enhancers and promoters operably linked to for example, SEQ ID NO: 1.

Other viral vectors that might be used in the invention are adeno-associated virus (AAV)-based vectors. AAV-based vectors are advantageous because they exhibit high transduction efficiency of target cells and can integrate into the host genome in a site-specific manner. Use of recombinant AAV vectors is discussed in detail in Tal, J., J. Biomed. Sci. 7:279-291, 2000 and Monahan and Samulski, Gene Therapy 7:24-30, 2000. A preferred AAV vector comprises a pair of AAV inverted terminal repeats which flank at least one cassette containing a tissue (e.g., gum)- or cell-specific promoter operably linked to a urease nucleic acid. The DNA sequence of the AAV vector, including the LTRs, the promoter and, for example, urease gene may be integrated into the host genome.

The use of herpes simplex virus (HSV)-based vectors is discussed in detail in Cotter and Robertson, Curr. Opin. Mol. Ther. 1:633-644, 1999. HSV vectors deleted of one or more immediate early genes (IE) are advantageous because they are generally non-cytotoxic, persist in a state similar to latency in the host cell, and afford efficient host cell transduction. Recombinant HSV vectors can incorporate approximately 30 kb of heterologous nucleic acid. A preferred HSV vector is one that: (1) is engineered from HSV type I, (2) has its IE genes deleted, and (3) contains a tissue-specific promoter operably linked to a urease nucleic acid. HSV amplicon vectors may also be useful in various methods of the invention. Typically, HSV amplicon vectors are approximately 15 kb in length, and possess a viral origin of replication and packaging sequences.

Retroviruses such as C-type retroviruses and lentiviruses might also be used in the invention. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells. They are also highly efficient at transducing human epithelial cells. Lentiviral vectors for use in the invention may be derived from human and non-human (including SW) lentiviruses. Preferred lentiviral vectors include nucleic acid sequences required for vector propagation as well as a tissue-specific promoter operably linked to, for example, SEQ ID NO: 1, derivatives, variants and fragments thereof. These former may include the viral LTRs, a primer binding site, a polypurine tract, att sites, and an encapsidation site.

A lentiviral vector may be packaged into any suitable lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN), might also be used in the invention. Use of alphaviruses is described in Lundstrom, K., Intervirology 43:247-257, 2000 and Perri et al., Journal of Virology 74:9802-9807, 2000. Alphavirus vectors typically are constructed in a format known as a replicon. A replicon may contain (1) alphavirus genetic elements required for RNA replication, and (2) a heterologous nucleic acid such as one encoding SEQ ID NO: 1. Within an alphavirus replicon, the heterologous nucleic acid may be operably linked to a tissue-specific promoter or enhancer.

Recombinant, replication-defective alphavirus vectors are advantageous because they are capable of high-level heterologous (therapeutic) gene expression, and can infect a wide host cell range. Alphavirus replicons may be targeted to specific cell types by displaying on their virion surface a functional heterologous ligand or binding domain that would allow selective binding to target cells expressing a cognate binding partner. Alphavirus replicons may establish latency, and therefore long-term heterologous nucleic acid expression in a host cell. The replicons may also exhibit transient heterologous nucleic acid expression in the host cell. A preferred alphavirus vector or replicon is non-cytopathic.

In many of the viral vectors compatible with methods of the invention, more than one promoter can be included in the vector to allow more than one heterologous gene to be expressed by the vector. Further, the vector can comprise a sequence which encodes a signal peptide or other moiety which facilitates the secretion of a gene product from the host cell.

To combine advantageous properties of two viral vector systems, hybrid viral vectors may be used to deliver a nucleic acid to a target tissue. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral lilts may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the host cell genome and effect stable urease gene expression.

Other nucleotide sequence elements which facilitate expression of SEQ ID NO: 1, derivatives, variants and fragments thereof and cloning of the vector are further contemplated. For example, the presence of enhancers upstream of the promoter or terminators downstream of the coding region, for example, can facilitate expression.

In an aspect of the method wherein the viral expression vector is an AAV vector capable of transducing the target cell, the AAV vector is free of both wildtype and helper virus. Exemplary types of AAV vectors useful in the present invention include serotype 2 AAV vectors and chimeric serotype 1/2 AAV vectors.

2) Other Delivery Vehicles

Many nonviral techniques for the delivery of a nucleic acid sequence into a cell can be used, including direct naked DNA uptake (e.g., Wolff et al., Science 247: 1465-1468, 1990), receptor-mediated DNA uptake, e.g., using DNA coupled to asialoorosomucoid which is taken up by the asialoglycoprotein receptor in the liver (Wu and Wu, J. Biol. Chem. 262: 4429-4432, 1987; Wu et al., J. Biol. Chem. 266: 14338-14342, 1991), and liposome-mediated delivery (e.g., Kaneda et al., Expt. Cell Res. 173: 56-69, 1987; Kaneda et al., Science 243: 375-378, 1989; Zhu et al., Science 261: 209-211, 1993). Many of these physical methods can be combined with one another and with viral techniques; enhancement of receptor-mediated DNA uptake can be effected, for example, by combining its use with adenovirus (Curiel et al., Proc. Natl. Acad. Sci. USA 88: 8850-8854, 1991; Cristiano et al., Proc. Natl. Acad. Sci. USA 90: 2122-2126, 1993). Other examples include stem cells such as mesenchymal stem cells, hematopoietic stem cells, cardiac stem cells or neural stem cells, embryonic stem cells that have been engineered to express MCPIP. Such stem cells can be administered in such a way to be incorporated in to tissues of a patient in need. In a particular embodiment, stem cells are administered to myocardial tissue. Bu L, et al “Human ISL1 heart progenitors generate diverse multipotent cardiovascular cell lineages” Nature 2009 460:113-117.

Aspects of the invention therefore include polynucleotides encoding at least one mammalian MCPIP and amino acid sequences representing at least one MCPIP protein. Aspects of the invention also include subunits or variants of polynucleotides or MCPIP proteins or peptides encoded by those polynucleotides.

It is well known in the art that a single amino acid may be encoded by more than one nucleotide codon—and that the nucleotide sequence may be easily modified to produce an alternate nucleotide sequence that encodes the same peptide. Therefore, alternate embodiments of the present invention include alternate DNA sequences encoding peptides containing the amino acid sequences described for MCPIP. DNA sequences encoding peptides containing the claimed amino acid sequence include DNA sequences which encode any combination of the claimed sequence and any other amino acids located N-terminal or C-terminal to the claimed amino acid sequence.

It is to be understood that amino acid and nucleic acid sequences may include additional residues, particularly N- or C-terminal amino acids or 5′ or 3′ nucleotide sequences, and still be essentially as set forth in the sequences disclosed herein, as long as the sequence produces a functionally similar polypeptide or protein. A nucleic acid fragment of almost any length may be employed, and may be combined with other DNA sequences, such as promoters, polyadenylation signals, additional restriction enzyme sites, multiple cloning sites, other coding segments, and the like. Therefore, overall length may vary considerably.

MCPIP polypeptides, as used herein, may comprise short fragments of proteins often referred to as peptides, as well as longer fragments generally referred to as polypeptides, and full-length proteins. These polypeptides can be prepared by standard peptide synthesis methods known to those of skill in the art, but may also be produced using an expression vector having a polynucleotide sequence encoding the polypeptide(s) of choice operably linked to appropriate promoter, terminator, and other functional sequences (such as a sequence encoding a purification tag) to facilitate expression and purification of the peptides.

It is to be understood that amino acid and nucleic acid sequences may include additional residues, particularly N- or C-terminal amino acids or 5′ or 3′ nucleotide sequences, and still be essentially as set forth in the sequences disclosed herein, as long as the sequence confers MCP-1 inducible transcription factor activity upon the polypeptide or protein moiety of the expressed protein. Nucleic acids which hybridize with a nucleic acid encoding the amino acid sequence of SEQ ID NO: 2 or SEQ ID NO: 4 under stringent conditions and encode a polypeptide having a similar MCP-1 inducible transcription factor activity to that of a polypeptide comprising SEQ ID NO: 2 or SEQ ID NO: 4 are also included as embodiments of the present invention.

The term “moderately stringent conditions”, as used herein, means conditions in which non-specific hybridization will not generally occur. Hybridization under such conditions can be performed based on the description provided in Molecular Cloning: A Laboratory Manual 2nd ed., published by cold Spring Harbor Laboratory in 1989, edited by T. Maniatis et al. Typically, for stringent hybridization conditions a combination of temperature and salt concentration should be chosen that is approximately 12-20° C. below the calculated Tₘ of the hybrid under study. The Tₘ of a hybrid between a polynucleotide of interest or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.-16.6(log₁₀[Na⁺])+0.41(% G+C)−0.63(% formamide)-600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C. According to another example, stringent conditions include incubation with a probe in 6×SSC containing 0.5% SDS, 5×Denhardt's solution and 100 micrograms/ml salmon sperm DNA at 60 degrees C.

Additional nucleic acid bases may be added either 5′ or 3′ to the MCPIP ORF, and may be combined with other DNA sequences, such as promoters, polyadenylation signals, additional restriction enzyme sites, multiple cloning sites, other coding segments, and the like. Therefore, overall length of such a polynucleotide may vary considerably. In a method described by the present invention, a nucleotide sequence of SEQ ID NO: 1 is inserted into a protein expression vector to produce a protein which can be used to synthesize a DNA copy of an RNA molecule. The DNA can then be amplified to form multiple copies.

“Control sequences” are those DNA sequences that are necessary for the expression of a protein from a polynucleotide sequence containing such a sequence, operably linked to the polynucleotide sequence encoding the protein. These sequences include prokaryotic sequences such as, for example, promoters, operators, and ribosome binding sites, and eukaryotic sequences such as, for example, promoters, enhancers, and polyadenylation signals. “Expression systems” are DNA sequences (such as, for example, plasmids) appropriate for expression of a target protein in a particular host cell, these sequences comprising appropriate control sequences for protein expression in the host cell operably linked to the polynucleotide sequence encoding the target protein.

It is to be understood that a “variant” of a polypeptide is not completely identical to the native protein. A variant MCPIP protein, for example, can be obtained by altering the amino acid sequence by insertion, deletion or substitution of one or more amino acids. The amino acid sequence of the protein can be modified, for example, by substitution to create a polypeptide having substantially the same or improved qualities as compared to the native polypeptide. The substitution may be a conserved substitution. A “conserved substitution” is a substitution of an amino acid with another amino acid having a side chain that is similar in polar/nonpolar nature, charge, or size. The 20 essential amino acids can be grouped as those having nonpolar side chains (alanine, valine, leucine, isoleucine, proline, phenylalanine, and tryptophan), uncharged polar side chains (methionine, glycine, serine, threonine, cystine, tyrosine, asparagine and glutamine), acidic side chains (aspartate and glutamate), and basic side chains (lysine, arginine, and histidine). Conserved substitutions might include, for example, Asp to Glu, Asn, or Gln; His to Lys, Arg or Phe; Asn to Gln, Asp or Glu; and Ser to Cys, Thr or Gly. Alanine, for example, is often used to make conserved substitutions.

To those of skill in the art, variant polypeptides can be obtained by substituting a first amino acid for a second amino acid at one or more positions in the polypeptide structure in order to affect biological activity. Amino acid substitutions may, for example, induce conformational changes in a polypeptide that result in increased biological activity.

Those of skill in the art may also make substitutions in the amino acid sequence based on the hydrophilicity index or hydropathic index of the amino acids. A variant amino acid molecule of the present invention, therefore, has less than one hundred percent, but at least about fifty percent, and preferably at least about eighty to about ninety percent amino acid sequence homology or identity to the amino acid sequence of a polypeptide comprising SEQ ID NO: 2, or a polypeptide encoded by SEQ ID NO: 4. Therefore, the amino acid sequence of the variant MCPIP protein corresponds essentially to the native MCPIP protein amino acid sequence. As used herein, “corresponds essentially to” refers to a polypeptide sequence that will elicit a similar biological and enzymatic activity to that generated by a MCPIP protein comprising SEQ ID NO 2 or SEQ ID NO: 4, such activity being at least about 70 percent that of the native MCPIP protein, and more preferably greater than 90 percent of the activity of the native MCPIP protein.

A variant of the MCPIP protein may include amino acid residues not present in a corresponding MCPIP protein comprising SEQ ID NO 2, or may include deletions relative to the MCPIP protein comprising SEQ ID NO 2. A variant may also be a truncated “fragment,” as compared to the corresponding protein comprising SEQ ID NO 2, the fragment being only a portion of the full-length protein.

Polypeptides of the invention may be delivered to a cell via attachment of one or more polypeptides to cell permeable, or “importation competent” signal peptide sequences, and membrane translocation sequences that have been shown to facilitate the transport of attached peptides and proteins into cells. Several sequences of this kind have previously been described, including the hydrophobic region of the signal sequence of Kaposi fibroblast growth factor which has been fused to the nuclear localization sequence (NLS) of p50 to produce the peptide known as SN50 (U.S. Pat. No. 5,807,746, Lin et al.). Polypeptides may also be delivered via a membrane translocating sequence described in U.S. Pat. Nos. 6,248,558; 6,432,680; and 6,780,843 (Rojas et al.). MCPIP, or a nuclear localization sequence that blocks nuclear localization of MCPIP, may also be delivered via the cell-permeable sequence described in U.S. Patent Application Number 20060099275 (Lin and Budu). Other membrane-translocating sequences are also well-known to those of skill in the art. Non-invasive delivery of proteins via membrane translocating peptides is discussed by Hawiger in Curr. Opin Chem. (1999) 3: 89-94, and multiple examples of both in vitro and in vivo use of membrane translocation via cell-permeable peptide sequences are available in the literature. The HIV-Tat peptide, for example, has been used in a number of studies to deliver cargo peptides to target cells (Ribeiro, M. M., et al. Biochem. Biophys. Res. Commun. (2003) 305(4): 876-81; Jung, H. J., et al. Biochem. Biophys. Res. Commun. (2006) 345(1): 222-228; Barnett, E. M., et al. Invest. Opthalmol. Vis. Sci. (2006) 47(6): 2589-2595; Hogue, M., et al. J. Biol. Chem. (2005) 280(14): 13648-13657; Mondal D., et al. Exp. Biol. Med. (2005) 230(9): 631-644; Kittiworakam, J., et al. J. Biol. Chem. (2006) 281(6): 3105-3115).

Polynucleotides encoding all or a part of the amino acid sequence of MCPIP may be delivered in vitro or in vivo by a variety of means known to those of skill in the art, such as, for example, viral gene delivery, naked DNA, delivery via cationic lipid carriers, and plasmid DNA/polylysine complexes.

As used herein, MCPIP polypeptides include variants or biologically active fragments of the peptides, as well as peptides which may contain additional amino acids either N-terminal or C-terminal (or both) to the disclosed sequences, their derivatives, variants, or functional counterparts. A “functional counterpart” can include, for example, a peptide nucleic acid (PNA). A “variant” of the peptide is not completely identical to a disclosed MCPIP polypeptide sequence. A variant, given the disclosure of the present invention, can be obtained by altering the amino acid sequence by insertion, deletion or substitution of one or more amino acid. The amino acid sequence of a disclosed peptide can be modified, for example, by substitution to create a peptide having substantially the same or improved qualities. The substitution may be a conserved substitution. A “conserved substitution” is a substitution of an amino acid with another amino acid having a side chain that is similar in polar/nonpolar nature, charge, or size. The 20 essential amino acids can be grouped as those having nonpolar side chains (alanine, valine, leucine, isoleucine, proline, phenylalanine, and tryptophan), uncharged polar side chains (methionine; glycine, serine, threonine, cysteine, tyrosine, asparagine and glutamine), acidic side chains (aspartate and glutamate) and basic side chains (lysine, arginine, and histidine). Conserved substitutions might include, for example, Asp to Glu, Asn or Gln; His to Lys, Arg or Phe; Asn to Gln, Asp or Glu, Leu to De or Val, and Ser to Cys, Thr or Gly. Alanine is commonly used to make conserved substitutions.

To those of skill in the art, variant polypeptides can be obtained by substituting a first amino acid for a second amino acid at one or more positions in the peptide structure in order to affect biological activity. Amino acid substitutions may, for example, induce conformational changes in a polypeptide that result in increased biological activity. Those of skill in the art may also make substitutions in the amino acid sequence based on the hydrophilicity index or hydropathic index of the amino acids.

A variant polypeptide of the present invention has less than 100%, but at least about 50%, and more preferably at least about 80% to about 90% amino acid sequence homology or identity to the amino acid sequence of a corresponding native nucleic acid molecule or polypeptide comprising SEQ ID NO 1, SEQ ID NO 2, SEQ ID NO 3, or SEQ ID NO 4. The amino acid sequence of a variant MCPIP polypeptide therefore corresponds essentially to the disclosed amino acid sequences. As used herein, “corresponds essentially to” refers to a polypeptide sequence that will elicit a similar biological activity as that generated by a disclosed MCPIP, such activity being from at least about 70 percent of that of disclosed MCPIP polypeptide, to greater than 100 percent of the activity of a disclosed MCPIP peptide.

The human MCPIP polypeptide sequence comprises a Zc3h12a Ribonuclease domain at residues 130-280 and zinc finger domain at 284-309. It is to be understood that non-conserved substitutions at these regions will likely result in negating activity of the protein. Mizgalska et al., FEBS Journal 276:7386-7399 (2009). Conserved substitutions in these domains will likely result in preserving activity of the protein. Most mutations, whether conservative or non-conservative, outside the two domains will not likely affect activity of the protein to any great extent. Accordingly, the disclosure of SEQ ID NO: 2 combined with the knowledge in the art regarding the genetic code establishes possession of using the genus of nucleic acid sequences that encode SEQ ID NO. 2, as well as sequences that encode a polypeptide with at least 85 percent sequence identity with SEQ ID NO. 2.

A variant of a disclosed MCPIP may include amino acid residues not present in the corresponding MCPIP, or may include deletions relative to the corresponding MCPIP. A variant may also be a truncated “fragment” as compared to the corresponding MCPIP, i.e., only a portion of the amino acid sequence of certain disclosed MCPIPs. The fragment may be at least 20, 30, 40, 50, 60, 80, 90, 100, 150, 175, 200, 250, 300, 350, 400, 450, 500, 550, 560, 570, 580, 585, 590, or 595 amino acids in length.

Variants, including fragments, possessing activity of MCPIP, refer to those, that are capable of degrading interleukin 1-beta (see Mizgalska et al.). Alternatively, the variant has activity according to the in vitro assay for transcription factor activity as taught in Zhou et al., Circulation Research, 98:1177-1185 (2006). Preferably, naturally or non-naturally occurring polypeptide variants have amino acid sequences which are at least about 55, 60, 65, or 70, preferably about 75, 80, 85, 90, 96, 96, or 98% identical to the full-length amino acid sequence or a fragment thereof. Percent identity between a putative polypeptide variant and a full length amino acid sequence is determined using the Blast2 alignment program (Blosum62, Expect 10, standard genetic codes).

### EXAMPLES

### Example 1

### Characterization of Transgenic Mice that Express MCPIP Selectively in the Myocardium

To test for the possible protective function of MCPIP expression in the myocardium, we generated transgenic mice line with full-length murine MCPIP gene under the control of the α-MHC promoter [FIG. 1A], as we had previously done to generate MCP-1 transgenic mice (24). Three lines of MCPIP TG mice were confirmed by PCR analysis. These transgenic mice displayed no abnormalities, and their external appearance was indistinguishable from that of nontransgenic controls, and the mice have normal life expectancies when grown in. To test for the tissue-specific expression of MCPIP gene, protein isolated from different tissues of TG mice was analyzed by immunoblot using anti-MCPIP antibody. We found a marked expression of MCPIP protein in the heart tissue, but it was not detectable in the other tissues such as liver, brain, and skeletal muscle [FIG. 1B]. Immunoperoxidase staining also confirmed that MCPIP expression was localized to the cardiomyocytes [FIG. 1C]. Among the three lines of TG mice we generated, one line (Line 20) that showed highest level of MCPIP expression and stable inheritance of the transgene in the myocardium was used for further experiments [FIGS. 1D,E]. Hearts of the TG mice showed no evidence of cardiac morphological defects. Histological examination of the hearts from 12-week-old mice demonstrated no myocardial disarray, cellular infiltration, necrosis, or fibrosis under basal conditions (data not shown). There were no detectable differences in heart weight and echocardiographic parameters between TG mice and age-matched nontransgenic litter mates [Table].

### Example 2

### MCPIP Gene Expression in the Heart after LPS Exposure

MCPIP expression is induced in almost all eukaryotic cell lines which are exposed to LPS (20, 21). To determine the expression changes of MCPIP in the myocardium after LPS exposure, we challenged WT and age-, sex-marched TG mice with LPS and tested cardiac tissues for MCPIP expression at transcript and protein levels by real-time PCR and immunoblot analysis, respectively. Expression of MCPIP in the myocardium of TG mice was not affected by the treatment of LPS, whereas expression of MCPIP in the myocardium of WT mice was significantly increased in the early time point of LPS treatment (3 hr after LPS injection) compared to the untreated WT controls, followed by a decrease in MCPIP expression by 18 hr after LPS treatment [FIG. 2]. These results were consistent with the previous report showing that MCPIP belongs to the group of early-response genes (21).

### Example 3

### Cardiac-Targeted Expression of MCPIP Attenuates LPS-Induced Myocardial Dysfunction

To investigate whether cardiac dysfunction differs between WT and age-, sex-matched TG mice after LPS challenge, a serial in vivo echocardiography was performed to assess left ventricular structure and function before and 18 hr after LPS injections. There were no detectable differences in echocardiographic parameters between TG mice and age-, sex-matched WT mice at the beginning of the experiments (data not shown). FIG. 3A shows representative M-mode echocardiographies of WT and TG mice at 18 hours after administration of saline or 10 mg/kg of LPS. At this LPS concentration, WT mice hearts showed a 12% increase in left ventricular end-systolic diameters (ESD) and a 26% decrease in the percentage of fractional shortening (FS %) (FIGS. 3B and C), indicating a decrease in myocardial contractility in WT mice after LPS challenge. In contrast, TG mice hearts showed much less increase in left ventricular ESD and only a slight decrease in FS % after LPS challenge (FIGS. 3B and C), demonstrating that cardiac-targeted expression of MCPIP protected against LPS-induced myocardial dysfunction.

### Example 4

### Cardiac-Targeted Expression of MCPIP Inhibits LPS-Induced Production of Inflammatory Cytokines TNF-α, IL-1β, and IL-6 in the Myocardium

To determine whether protection against LPS-induced myocardial dysfunction by cardiac-targeted expression of MCPIP is associated with inhibition of LPS-induced inflammatory cytokine production in the myocardium, we evaluated myocardial TNF-α, IL-1β and IL-6 expression in LPS-treated WT and TG mice by measurement of their transcript and protein levels. In LPS-treated WT mice, the levels of TNF-α, IL-1β and IL-6 mRNA levels were markedly increased in the myocardium after 18 hr of LPS exposure in comparison with those of vehicle-treated mice (FIG. 4A-C). This increase was significantly attenuated in the myocardium of LPS-treated TG mice, and the levels reached almost exactly the levels found in vehicle-treated controls (FIG. 4A-C). This pattern of gene expression changes was also seen at the protein level (FIG. 4D-F), suggesting that cardiac-targeted expression of MCPIP leads to suppression of myocardial inflammatory responses during endotoxaemia. We further examined the effects of cardiac-targeted expression of MCPIP on LPS-induced systemic inflammatory response by measurement of the levels of circulating inflammatory cytokines and chemokines in the serum. In LPS-treated WT mice, the levels of TNF-α, IL-1β, IL-6, INF-γ, MCP-1, and IL-10 increased in the serum after 18 hours of LPS exposure (FIG. 5A-F). In LPS-treated TG mice, the increase in serum TNF-α, IL-1β, IL-6, INF-γ, and MCP-I levels was significantly less than that with LPS-treated WT mice (FIG. 5A-E). Moreover, we detected a significant stimulatory effect of MCPIP expression on IL-10 levels in the serum from LPS-treated TG mice (FIG. 5F). Taken together, these data suggest that cardiac-targeted expression of MCPIP suppressed LPS-induced increases in inflammatory cytokines in the myocardium and serum.

### Example 6

### Cardiac-Targeted Expression of MCPIP Attenuates LPS-Induced iNOS Expression and Peroxynitrite Formation in the Myocardium

There is much evidence that high levels of nitric oxide (NO) produced by inducible nitric oxide synthase (iNOS) contribute to the systemic hypotension and myocardial dysfunction in sepsis (25-27). To determine the mechanism by which cardiac-targeted expression of MCPIP preserves cardiac function, we measured myocardial iNOS expression, NO formation as nitrite/nitrate accumulation in the serum and heart homogenates. Real-time PCR analysis showed an increase in iNOS transcript levels in the myocardium of WT mice 18 hr after LPS exposure in comparison with the vehicle-treated mice (FIG. 6A). In contrast, this increase in iNOS mRNA levels was significantly less in the myocardium from LPS-treated TG mice (FIG. 6A). We performed immunohistochemical staining of cardiac sections from WT and TG mice to examine the levels of iNOS protein expression. iNOS positive cells were not detectable in cardiac sections from vehicle-treated mice, but cardiac sections from LPS-treated WT mice showed strong immunostaining for iNOS (FIG. 6B). However, cardiac sections from LPS-treated TG mice showed much less staining for iNOS (FIG. 6B). Strong iNOS staining was mainly localized to cardiac myocytes in LPS-treated WT heart, and quantification of the staining revealed a drastic reduction in iNOS staining in the myocardium of the LPS-treated TG mice as compared with LPS-treated WT mice (FIGS. 6B, C). Cardiac-specific expression of MCPIP also inhibited iNOS activity, as indicated by the reduced plasma and cardiac levels of nitrite/nitrate in LPS-treated TG mice (FIGS. 6D, E). Compared with vehicle treated mice, LPS-treated WT mice showed a ˜3-fold increase in plasma level and ˜2-fold increase in cardiac level of nitrite/nitrate, and these increase were much less in LPS-treated TG mice (FIGS. 6D, E).

NO interacts with superoxide to form peroxynitrite (ONOO⁻), a strong oxidative/nitrative molecule that plays a causative role in septic myocardial dysfunction (28). Therefore, we examined the production of peroxynitrite by testing for the presence of nitrotyrosine residues on proteins by immunohistochemistry with anti-3-nitrotyrosine (3-NT) antibody. As shown in FIG. 6F, there was no positive staining for nitrotyrosine in cardiac sections from vehicle-treated WT or TG mice, but cardiac sections from LPS-treated WT mice showed intense staining for 3-NT. The cardiac sections from LPS-treated TG mice showed much less staining for 3-NT. Quantification of the immunostaining revealed a significant reduction in 3-NT staining in the myocardium of LPS-treated TG mice as compared with LPS-treated WT mice (FIG. 6G). Collectively, these results demonstrate that cardiac-targeted expression of MCPIP inhibits LPS-induced myocardial iNOS expression and activity, which results in decreased cardiac peroxynitrite formation in the myocardium.

### Example 7

### Cardiac-Targeted Expression of MCPIP Inhibits LPS-Induced Apoptosis and Regulates Apoptosis-Related Gene Expression in the Myocardium

Apoptosis has recently been implicated in endotoxin-induced myocardial dysfunction (29-31). To assess whether the observed myocardial protection by cardiac-targeted expression of MCPIP was accompanied by protection from LPS-induced apoptosis in the myocardium, TUNEL staining was performed in cardiac tissues. In cardiac sections from vehicle-treated mice, a few TUNEL-positive cells were detected (FIG. 7A). In contrast, a much larger number of TUNEL-positive cells were observed in cardiac sections from LPS-treated WT mice hearts, whereas, much less TUNEL-positive cells were found in cardiac sections from LPS-treated TG mice (FIG. 6A). Quantification of TUNEL-positive cells in cardiac sections demonstrated that cardiac overexpression of MCPIP was significantly more resistant to LPS-induced apoptosis than that of WT animals (FIG. 7B).

Activation of caspase-3, which results in cleavage of poly (ADP-ribose) polymerase (PARP) into two fragments, i.e. p85 and p25, represents one of the key executioners of apoptosis (32). Detection of caspase-3 cleavage fragments of PARP has been established as a hallmark of apoptosis. As shown in FIG. 7C, analysis of caspase-3/7 activity in heart homogenates revealed that LPS induced a significant increase of caspase-3/7 activities in WT mice hearts. The increased caspase-3/7 activities in LPS-treated WT hearts coincided with the stronger intense band of the PARP p85 fragments, as detected by immunoblot (FIG. 7D). In contrast, cardiac-targeted expression of MCPIP significantly inhibited LPS-induced increase in caspase-3/7 activities and PARP cleavage in TG mice hearts (FIG. 7D). To further elucidate the mechanism by which cardiac-specific overexpression of MCPIP protects cell from LPS-induced apoptosis, we examined the expression changes of apoptosis-related genes, including antiapoptotic factors, cIAP and Bcl-2, and proapoptotic factors, Fas and FasL, in the myocardium after 18 hr of LPS exposure. Immunoblot analyses revealed that LPS exposure increased expression of pro-apoptotic protein Fas and FasL, and reduced expression of anti-apoptotic proteins cIAP and Bcl-2, respectively, in WT mice hearts. In contrast, cardiac-targeted expression of MCPIP preserved expression of cIAP and Bcl-2, and decreased expression of Fas and FasL, in TG hearts after LPS exposure, suggesting that cardiac-specific overexpression of MCPIP partially reverses LPS-induced imbalance of pro-apoptotic to anti-apoptotic proteins (FIGS. 7F,G).

### Example 8

### Cardiac-Targeted Expression of MCPIP Blocks LPS-Induced NF-κB Signaling by Inhibition of IKK Activation in the Myocardium

Activation of NF-κB pathway is thought to be a key signaling event involved in the pathogenesis of sepsis and septic myocardial dysfunction (7, 8). To determine whether cardiac-targeted expression of MCPIP inhibits LPS-induced cardiac NF-κB activation, we first examined p65 translocation to the nucleus and NF-κB p65 DNA binding activity by immunoblot and ELISA, respectively. Translocation of p65 from the cytosol into nucleus was evident in WT mice hearts after 18 hr of LPS exposure, whereas such nuclear translocation was attenuated in LPS-treated TG mice hearts (FIGS. 8A, B). The DNA binding activity of NF-κB p65 subunit in nuclear extracts was also significantly increased in LPS-treated WT mice hearts; whereas such increase in NF-κB p65 target DNA-binding activity in the nuclear fractions was markedly attenuated in LPS-treated TG heart (FIG. 8C).

A crucial step in the activation of NF-κB is the phosphorylation of IκB by the IκB kinase (IKK) complex (9, 10). The IKK complex consists of two highly homologous kinase subunits, IKKα and IKKIβ, and a nonenzymatic regulatory component, IKKγ/NEMO. To determine at which step in the pathway of NF-kB activation MCPIP exerts its inhibitory action, phosphorylation and cytosolic levels of IκBα were determined by immunoblot analysis (FIG. 8D-F), LPS treatment caused increased phosphorylation of IκBα in the heart homogenates from WT mice, and this phosphorylation was blocked in the hearts of LPS-treated TG mice. Because phosphorylation of IκBα is via activation of IKK complex, we then examined whether blockade of IκBα phosphorylation by MCPIP is due to its action on the IKK complex. As shown in FIG. 8G, immunoblot analysis showed that LPS treatment resulted in phosphorylation of IKKα and IKKβ in heart homogenates of WT mice, and this phosphorylation was inhibited by MCPIP expression in the TG mice (FIG. 8H), whereas the IKKα and IKKβ protein levels were not affected by cardiac-specific overexpression of MCPIP (Fig. G, I). These results indicate that cardiac-targeted expression of MCPIP suppresses LPS-induced cardiac NF-κB activation through inhibition of IKK complex activation.

### Discussion for Examples 1-8

In the examples above, the inventors demonstrated for the first time that cardiac-targeted expression of MCPIP can prevent endotoxin-induced myocardial dysfunction in experimental mice model of sepsis induced by administration of LPS. This myocardial protective effect by MCPIP is associated with reduced production of pro-inflammatory cytokines, decreased expression of iNOS and formation of peroxynitrite, as well as attenuated apoptosis signaling in the myocardium by inhibition of cardiac NF-κB signaling cascade. This is the first report of a genetic approach to dissect out the functional significance of MCPIP in sepsis-induced cardiac dysfunction.

The inventors observed a substantial increase in the levels of TNF-α, IL-1β, and IL-6 in the myocardium of WT mice after LPS exposure, which likely contribute to the myocardial depression in LPS-treated WT animals. The inventors also found that elevation of TNF-α, IL-1β, and IL-6 levels resulting from LPS exposure suppressed significantly due to the presence of MCPIP and thus resulting in preservation of myocardial function. This suggests that cardiac-targeted expression of MCPIP represents an inhibitory signal for LPS-induced NF-κB activation, because expression of these cytokines is classically regulated by NF-κB activation. This observation is consistent with previous findings that enhanced expression of MCPIP inhibited LPS-induced promoter activity of TNF-α (20), and regulated IL-1β and IL-6 mRNA stability (22, 23). The inventors also detected lowering of TNF-α, IL-1β, IL-6, INF-γ, and MCP-1 levels in serum in MCPIP TG mice compared with WT mice after LPS treatment. Moreover, an increased level of the anti-inflammatory cytokine IL-10 was observed in serum in MCPIP TG mice after 18 hr of LPS exposure. Thus, the modulatory effect of MCPIP on pro- and anti-inflammatory cytokines may provide one explanation for the protection of cardiac function in MCPIP TG mice.

The generation of reactive free radical, NO, has been well documented in the pathogenesis of myocardial dysfunction caused by LPS or sepsis (25-28). Specifically, LPS-dependent NF-κB-mediated iNOS expression has been described as being responsible for the overproduction of NO that mostly reacts with superoxide anion (O2-), leading to formation of peroxynitrite anion (ONOO⁻). Increasing evidence suggests that the harmful effects of NO in the heart and in the vasculature are due to the formation of ONOO⁻ that causes cellular lipid peroxidation, protein oxidation, and the nitration of tyrosine residues within proteins (26, 28, 34). The most abundant proteins for nitration modification within myocardium are actin and myosin, which have been found to be responsible for cardiac dysfunction (31, 35). In the present study, we found increased iNOS expression, elevation of nitrite and nitrate, and formation of peroxynitrite in the myocardium of WT mice after 18 hr of LPS exposure. These findings are in accordance with the previous reports that NO-derived oxidative species and particularly peroxynitrite are formed during LPS induced myocardial dysfunction (28). These changes that occurred in the myocardium of WT mice were attenuated by cardiac-targeted expression of MCPIP, resulting in protection of the heart against the toxic effect of LPS. These data suggest that cardiac-specific expression of MCPIP could inhibit LPS-induced cardiac iNOS expression and thus protects cardiac tissues from nitrosative stress during sepsis. This observation is consistent with our previous findings that enhanced expression of MCPIP inhibited LPS-induced promoter activity of iNOS (20).

Apoptosis has been described as a dominant feature of septic shock in patients and animal models (36, 37), and has recently been recognized as a major contributor in endotoxin-induced myocardial dysfunction (30, 38, 39). Here we showed that inhibition of LPS-induced apoptotic cell death in the myocardium is one of the consequences of cardiac-targeted expression of MCPIP. The TUNEL assay of apoptotic cells showed that enhanced expression of MCPIP decreases the LPS-induced apoptotic cell death in the myocardium. Caspase-3 represents one of the key executioners of apoptosis. Its activation is associated with significant impairment of cardiac contractility and its inhibition restores hemodynamic performance in certain models (30, 38). The caspase-3/7 activities and caspase-3-activated PARP cleavage were significantly increased in the myocardium of WT mice after 18 hr of LPS exposure, but markedly attenuated by the presence of MCPIP. The anti-apoptotic action of expression of MCPIP in heart could be mediated by the increased production of IL-10, because the anti-apoptotic activity of IL-10 has been documented in cardiomyocytes by the activation of extracellular signal-regulated kinase 1/2 (ERK 1/2) MAPK phosphorylation (40). On the other hand, the decreased levels of pro-inflammatory cytokines TNF-α, IL-1β, IL-6 and NO by cardiac-targeted expression of MCPIP could be promoting an anti-apoptotic effect, because these cytokines have been shown to promote cytosolic and mitochondrial reactive oxygen species (ROS) production that lead to apoptotic events in a variety of tissues (41-44). Even though there was no significant difference in expression of apoptotic genes in TG and WT mice under basal condition, we did find that expression of MCPIP in heart can maintain expression of anti-apoptotic protein, cIAP and Bcl-2, and decrease LPS-induced expression of pro-apoptotic proteins, Fas and FasL. These findings may account for the finding that TG hearts have lower rates of apoptosis and less myocardial dysfunction than WT hearts following LPS exposure. However, we can not rule out a direct action of MCPIP on expression of apoptosis-related proteins in sepsis at the present time.

As described above, the cardioprotective effects of enhanced expression of MCPIP in the myocardium reflects its suppression of the inflammatory signaling cascade. Mice genetically deficient in MCPIP also develop severe systemic inflammation early in life (22). NF-κB is a transcription factor that regulates gene transcription of many pro-inflammatory cytokines, and has been considered as a pathological mechanism of septic multiple organ dysfunctions (7, 8). It is therefore logical that suppression of cardiac NF-κB activity can be a potential mechanism for regulating myocardial inflammatory responses by cardiac-targeted expression of MCPIP. The major mechanism of NF-κB activation involves phosphorylation/activation of inhibitor kappa B kinase (IKK), which leads to the degradation of IκB and in turn translocation of NF-κB to the nucleus and its activation (9, 10). The requirement and involvement of IKK in the NF-κB pathway activation has been documented in various cardiac pathologic conditions [45, 46]. The present finding that expression of MCPIP in the myocardium attenuated LPS-induced the IKKα/β phosphorylation resulted in blockage of IκBα phosphorylation, and p65 subunit translocation in the myocardium suggests that MCPIP may be involved the inhibition of IKK activation. This reduced IKK activation in the myocardium might be responsible for alleviation of LPS-induced NF-kB activation. Although the phosphorylation of IKKα/β decreased in the myocardium of TG mice after LPS exposure, the molecular mechanism by which cardiac-specific expression of MCPIP inhibits LPS-induced activation of IKK complex remains to be elucidated. This reduced activation of IKKα/β can not be attributed to differences in the amount of kinase, because the levels of IKK determined by immunoblot were similar. The multiple upstream kinases, such as transforming growth factor β-activating kinase 1(TAK1), mitogen-activated protein kinase/ERK kinase kinase 1 (MEKK1), and NF-κB-inducing kinase (NIK), reportedly regulate IKK phosphorylation/activation (47). In addition, mono-ubiquitylation of the IKKβ has bee shown to regulate its phosphorylation and persistent activation (48), and unanchored polyubiquitin chains can directly activate IKK complex (49). The exact molecular targets affected by MCPIP remain to be identified.

Regardless of the specific mechanisms involved, the present results revealed that suppression of cardiac NF-κB activation by enhanced expression of MCPIP in the myocardium decrease LPS-stimulated pro-inflammatory cytokine production, iNOS expression and nitrosative stress, and promotes preservation of cardiac function. Cardiac-targeted expression of MCPIP suppresses LPS-mediated NF-kB activation by blocking LPS-induced IκBα phosphorylation through inhibition of activation of IKK complex. These findings provide the first in vivo evidence that MCPIP is a potential anti-inflammatory mediator, which may have therapeutic values to protect heart from inflammatory pathologies.

### Materials and Methods Related to Examples 1-8

Generation of TG mice. The transgene was designed as we previously described (24). In short, to express murine MCPIP gene in the myocardium of FVB/N mice, the full-length (1.7 kb) sequence of murine MCPIP cDNA was cloned into pBS40 construct (24). This construct was amplified by PCR and sequenced to confirm lack of mutations, and ligated to the 5.5-kb mouse α-myosin heavy chain (MHC) promoter of pBS40 construct at SalI and KpnI restriction sites. The transgene sequent was purified from the plasmid sequences by restriction digestion and gel electrophoresis. Three transgenic founder lines were produced by microinjecting MCPIP transgene into the pronuclei of FVB/N zygotes. Transgenic founders and offspring were identified and genotyped by PCR on mouse tail DNA using the primers: MCPIP primer, CAGGACACTGTGGATCTCAG, and MHC primer, TTCTCTGCCCAGCTGCCC. Transgenic mice were bred with nontransgenic FVB/N mice and the homozygous TG mice were produced by interbreeding and maintained in our animal facility.

Experimental animal model. Experimental procedures in mice and protocol used in this study were approved by Animal Care and Use Committee of the University of Central Florida, in accordance with the Guide for the Care and Use of Laboratory Animals published by the US National Institutes of Health (NIH Publication No. 86-23, Revised 1996). Adult male wild-type (WT) mice (10-12 wk of age, FVB/N strain) purchased from Harlan Laboratories (Indianapolis, Ind.) and age-, sex-matched homozygous MCPIP TG mice were used. Animals were injected intraperitoneally with 10 mg/kg Escherichia coli LPS (0111:B4; Sigma) dissolved in sterile saline (LPS-treated groups) or with an equivalent volume of pathogen-free saline only (vehicle-treated groups). The dosage of LPS was based on the previous report that showed cardiac dysfunction without any mortality (50). Animals were exposed to 12 hr light/12 hr dark cycle with free access to food and tap water. At 18 hr post-injection, the hearts were removed under isoflurane anesthesia after echocardiographic measurements and blood sample collections, washed in cold saline and fixed in 10% phosphate-buffered formaldehyde for histology or frozen in liquid nitrogen for RNA and protein analysis.

Determination of cardiac dysfunction by echocardiography. At the beginning of LPS treatment and on the day that animals were euthanized, cardiac function was assessed by echocardiography. In brief, mice were lightly anesthetized via a nose cone and maintained with 0.5 to ˜2.0% isoflurane (AErrane; Baxter, McGaw Park, Ill.) mixed with oxygen. The chest was shaved, and animals were placed in a supine position with a slight tilt to the left decubitus position. Thermoregulation was achieved by using an autoregulated heating pad. A 15-MHz high-frequency transducer connected to an Agilent Technologies SONOS 4500 ultrasound machine (Philips Medical System; Agilent Technologies, Santa Clara, Calif.) was used. A two-dimensional short-axis view of the left ventricle was obtained at the level of the papillary muscles, and two-dimensionally targeted M-mode tracings were recorded at a sweep speed of 100 mm/s. The left ventricular end-diastolic dimension (LVEDD) and left ventricular end-systolic dimension (LVESD) were measured using online analyzing system to calculate the left ventricular fractional shortening, an index of cardiac function, by the equation: fractional shortening=[(LVEDD−LVESD)/LVEDD]×100%. All measurements were made according to the guidance of the American Society of Echocardiography leading edge-to-leading edge technique. Data from three to five consecutive selected cardiac cycles were analyzed and averaged.

Histology and immunohistochemistry. The hearts fixed in 10% phosphate-buffered formaldehyde were routinely processed and paraffin-embedded. Sections (5 μm) were prepared from heart of each group of mice and stained with hematoxylin and eosin (HE) using standard protocols for histopathological analysis. Immunohistochemistry was performed on sections treated with 3% H₂O₂/methanol solution to quench endogenous peroxidase activity and permeabilized with proteinase K, and blocked with blocking buffer to block non-specific binding. Sections were then incubated with polyclonal rabbit anti-iNOS, anti-3-nitrotyrosine (3-NT, Upstate Biotechnology) or polyclonal rabbit anti-NFκB p65 (Santa Cruz Biotechnology) antibodies, respectively, overnight at 4° C. followed by incubation with horseradish peroxidase (HRP)-conjugated goat anti-rabbit antibody (Santa Cruz, Calif.). Peroxidase activity was visualized with diaminobenzidine (DAB). The immunoreactivity for iNOS and 3-NT were quantified, as described previously (51), from five randomly selected sections for each animal, and five animals were studied per group.

TdT-mediated dUTP nick-end-labeling (TUNEL) assay. Cleavage of genomic DNA during apoptosis was detected by TUNEL assay using in situ Cell Death Detection Kit, TMR red (Roche Diagnostics) and performed according to the manufacturer's instructions. Briefly, sections were treated with 2% H₂O₂/methanol solution to quench endogenous peroxidase activity and permeabilized with proteinase K, then incubated with labeling buffer containing terminal deoxynucleotidyl transferase (TdT) and TMR red-labeled dUTP at 37° C. for 60 min. After wash, the slides were mounted and the signals were visualized with TMR red under a fluorescent microscope. The total TUNEL-positive cells were calculated in five randomly selected fields for each animal and five animals were studied per group.

Quantitative real-time RT-PCR. Total RNA was extracted from frozen LV tissue using TRIzol reagent (Invitrogen) according to the manufacturer's instructions. RNA quality and quantity was monitored at 260 nm. Total RNA (1 μg) was reverse transcribed to cDNA using the SuperScript First-Strand Synthesis System (Bio-Rad). Real-time RT-PCR was performed with iCycler iQ real-time PCR detection system (Bio-Rad) using the Quantitect SYBR Green RT-PCR kit (Qiagen). Primer pairs used were as follows: MCPIP: 5′-TGAGCCATGGGAAGAAGGAAGTCT-3′ and reverse, 5′-TGTGCTGGTCTGTGATAGGCACAT-3′; TNF-α: forward 5′-GTGGAACTGGCAGAAGAGGC-3′ and reverse, 5′-AGACAGAAGAGCGTGGTGGC-3′; IL-1β: forward, 5′-GGAAGATTCTGAAGAAGAGACGG-3′ and reverse, 5′-TGAGATTTTTAGAGTAACAGG-3; IL-6: forward, 5′-ACAAGTCGGAGGCTT AATTACACAT-3′ and reverse, 5′-AATCAGAATTGCCATTGCACAA-3; and iNOS: forward, 5′-CGTCATTTCTGTCCGTCTCT-3′ and reverse, 5′-TTGCTGGCTGATGGCTGGCG-3′ β-actin, forward, 5′-ATGTTTGAGACCTTCAACA-3′ and reverse, 5′-CACGTCAGACTTCATGATGG-3′. Relative levels of mRNA transcripts for MCPIP, TNF-α, IL-1β, IL-6, and iNOS were normalized to β-actin expression by the method described previously [52].

Protein extracts and immunoblotting analysis. Heart samples were thawed and homogenized on ice in extraction buffer containing (in mmol/l) 25 Tris.HCl (pH 8.0), 150 NaCl, 15 KCl, 1 EDTA, and 1 DTT and 0.5% Triton X-100 and 5% glycerol, supplemented with protease, kinase, and phosphatase inhibitors, for total protein extraction. The separation of cytosolic and nuclear protein was carried out as previously described (50). Protein concentrations were determined by means of spectrophotometer assay (NanoDrop Technologies) to equalize the protein concentration of all samples.

Equal amounts of proteins were loaded and separated on 10% or 12% sodium dodecylsulfate polyacrylamide gels (Bio-Rad) and transferred onto nitrocellulose membranes (Amersham). The membranes were blocked for 1 h at room temperature in 5% non fat dry milk in Tris-buffered saline with 0.1% Tween 20 followed by incubation overnight at 4° C. with the respective primary antibodies: polyclonal rabbit anti-TNF-α, polyclonal rabbit anti-IL-1β, polyclonal rabbit anti-IL-6, polyclonal rabbit anti-PARP p85, polyclonal rabbit anti-Bcl-2, polyclonal rabbit anti-cIAP, polyclonal rabbit anti-Fas, anti-FasL, anti-NF-κB p65 (Santa Cruz Biotechnology), polyclonal rabbit anti-IκB-α, anti-phospho-IκB-α, anti-IKKα, anti-IKKβ, anti-phospho-IKKα/β, polyclonal rabbit anti-histone (Cell Signaling Technology), polyclonal goat anti-actin (Sigma). The immune complexes were detected autoradiographically using appropriate peroxidase-labeled secondary antibodies (Santa Cruz Biotechnology) and enhanced chemiluminescence detection reagent ECL (Amersham). Specific bands were quantified by densitometry using animaging software (Alphaimager 2200).

Determination of caspase-3/7 activity. The activity of caspases was determined using the APO-ONE Homogenous Caspase-3/7 Assay Kit (Promega) as per instructions from the manufacturer. Briefly, equal amounts of total myocardial proteins extracted from vehicle- and LPS-treated WT and MCPIP TG mice were diluted with phosphate-buffered saline (PBS) to a final volume of 50 μl. Then an equal volume of caspase substrate was added and samples were incubated at 37° C. for 1 hr. PBS was used as a blank. The fluorescence was measured at 485 nm with a Cary Eclipse (Varian) fluorescence spectrophotometer.

Quantification of NF-κB p65 activity. The NF-kB/p65 target DNA-binding activity was measured by using the colorimetric non-radioactive NF-κB p65 Transcript Factor Assay Kit (Cayman Chemical), an ELISA-based system that detects the binding of NF-κB to the immobilized DNA consensus binding site. The binding reaction was carried out overnight at 4° C. upon the addition of equal amounts of nuclear extracts and in the presence of an antibody directed against the NF-κB subunit p65. After incubation with HRP-conjugated secondary antibody and color development, the absorbance at 450 nm was measured with an ELISA microplate reader.

Biochemical measurements. Whole blood was collected from vehicle- or LPS-treated WT and MCPIP TG mice on the day that animals were euthanized. Cytokine (TNF-α, IL-1β, IL-6, IL-10, and IFN-γ) and chemokine (MCP-1) levels were measured in the serum with the Bio-Plex system using mouse multiple cytokines assay kits according to the manufacture's instructions. The levels of nitrite and nitrate, the major metabolites of NO, were measured in serum and heart homogenates by Griess reagent kit (Invitrogen) according to the manufacturer's instructions. The absorbance at 550 nm was measured using a microplate reader, and the concentration of nitrite and nitrate was determined from standard curves.

Statistical analysis. All values are presented as mean±standard error of the mean of n observations. n represents the number of animals studied. Significant differences between treatment mice groups were determined by one-way analysis of variance (ANOVA) followed by a Bonferroni post-test for multiple comparisons. A p-value of less than 0.05 was considered significant.

### Example 9

According to a further example, the subject invention relates to a method of ameliorating inflammation that is mediated by NF-κB activation. NF-κB activation has been shown to be involved in arthritis, glomerulonephritis, inflammatory colitis (that is typically marked by increased IL-1 expression), inflammatory airway diseases, such as asthma, and inflammation associated with atherosclerosis. As Table 1 (Tak and Firestein, J Clin Invest. 2001; 107(1):7-11) shows, NF-κB activation is also involved in these and other inflammatory diseases, such as Helicobacter pylori-associated gastritis, Inflammatory bowel disease and multiple sclerosis. Thus, according to another embodiment, the invention pertains to a method of treating an NF-κB activation inflammatory disease, by elevating MCPIP levels in a patient in need. Typically, elevating MCPIP levels is accomplished by directly administering MCPIP to said patient or administering a delivery vehicle comprising an expression construct encoding SEQ ID NO. 2 or a variant thereof possessing at least 90 percent identity and possesses MCPIP activity into said patient.

TABLE 1
NF-κB-activation inflammatory disease

Rheumatoid arthritis
Atherosclerosis
Multiple sclerosis
Chronic inflammatory demyelinating polyradiculoneuritis
Asthma
Inflammatory bowel disease
Helicobacter pylori-associated gastritis
Systemic inflammatory response syndrome

### REFERENCES

1. Martin, G. S., D. M. Mannino, S. Eaton, and M. Moss. 2003. The epidemiology of sepsis in the United States from 1979 through 2000. N Engl J Med 348:1546-1554.
2. Cohen, J. 2002. The immunopathogenesis of sepsis. Nature 420:885-891.
3. Krishnagopalan, S., A. Kumar, and J. E. Parrillo. 2002. Myocardial dysfunction in the patient with sepsis. Curr Opin Crit. Care 8:376-388.
4. Jean-Baptiste, E. 2007. Cellular mechanisms in sepsis. J Intensive Care Med 22:63-72.
5. Rudiger, A., and M. Singer. 2007. Mechanisms of sepsis-induced cardiac dysfunction. Crit. Care Med 35:1599-1608.
6. Knuefermann, P., J. Vallejo, and D. L. Mann. 2004. The role of innate immune responses in the heart in health and disease. Trends Cardiovasc Med 14:1-7.
7. Brown, M. A., and W. K. Jones. 2004. NF-kappaB action in sepsis: the innate immune system and the heart. Front Biosci 9:1201-1217.
8. Liu, S. F., and A. B. Malik. 2006. NF-kappa B activation as a pathological mechanism of septic shock and inflammation. Am J Physiol Lung Cell Mol Physiol 290:L622-L645.
9. May, M. J., and S. Ghosh. 1998. Signal transduction through NF-kappa B. Immunol Today 19:80-88.
10. Zhang, G., and S. Ghosh. 2000. Molecular mechanisms of NF-kappaB activation induced by bacterial lipopolysaccharide through Toll-like receptors. J Endotoxin Res 6:453-457.
11. Haudek, S. B., E. Spencer, D. D. Bryant, D. J. White, D. Maass, J. W. Horton, Z. J. Chen, and B. P. Giroir. 2001. Overexpression of cardiac I-kappaBalpha prevents endotoxin-induced myocardial dysfunction. Am J Physiol Heart Circ Physiol 280:H962-968.
12. Liu, S. F., X. Ye, and A. B. Malik. 1997. In vivo inhibition of nuclear factor-kappa B activation prevents inducible nitric oxide synthase expression and systemic hypotension in a rat model of septic shock. J Immunol 159:3976-3983.
13. Sheehan, M., H. R. Wong, P. W. Hake, V. Malhotra, M. O'Connor, and B. Zingarelli. 2002. Parthenolide, an inhibitor of the nuclear factor-kappaB pathway, ameliorates cardiovascular derangement and outcome in endotoxic shock in rodents. Mol Pharmacol 61:953-963.
14. Arnalich, F., E. Garcia-Palomero, J. Lopez, M. Jimenez, R. Madero, J. Renart, J. J. Vazquez, and C. Montiel. 2000. Predictive value of nuclear factor kappaB activity and plasma cytokine levels in patients with sepsis. Infect Immun 68:1942-1945.
15. Paterson, R. L., H. F. Galley, J. K. Dhillon, and N. R. Webster. 2000. Increased nuclear factor kappa B activation in critically ill patients who die. Crit. Care Med 28:1047-1051.
16. Cuenca, J., N. Goren, P. Prieto, P. Martin-Sanz, and L. Bosca. 2007. Selective impairment of nuclear factor-kappaB-dependent gene transcription in adult cardiomyocytes: relevance for the regulation of the inflammatory response in the heart. Am J Pathol 171:820-828.
17. Goren, N., J. Cuenca, P. Martin-Sanz, and L. Bosca. 2004. Attenuation of NF-kappaB signalling in rat cardiomyocytes at birth restricts the induction of inflammatory genes. Cardiovasc Res 64:289-297.
18. Hall, G., J. D. Hasday, and T. B. Rogers. 2006. Regulating the regulator: NF-kappaB signaling in heart. J Mol Cell Cardiol 41:580-591.
19. Zhou, L., A. Azfer, J. Niu, S. Graham, M. Choudhury, F. M. Adamski, C. Younce, P. F. Binkley, and P. E. Kolattukudy. 2006. Monocyte chemoattractant protein-1 induces a novel transcription factor that causes cardiac myocyte apoptosis and ventricular dysfunction. Circ Res 98:1177-1185.
20. Liang, J., J. Wang, A. Azfer, W. Song, G. Tromp, P. E. Kolattukudy, and M. Fu. 2008. A novel CCCH-zinc finger protein family regulates proinflammatory activation of macrophages. J Biol Chem 283:6337-6346.
21. Skalniak, L., D. Mizgalska, A. Zarebski, P. Wyrzykowska, A. Koj, and J. Jura. 2009. Regulatory feedback loop between NF-kappaB and MCP-1-induced protein 1 RNase. Febs J 276:5892-5905.
22. Matsushita, K., O. Takeuchi, D. M. Standley, Y. Kumagai, T. Kawagoe, T. Miyake, T. Satoh, H. Kato, T. Tsujimura, H. Nakamura, and S. Akira. 2009. Zc3h12a is an RNase essential for controlling immune responses by regulating mRNA decay. Nature 458:1185-1190.
23. Mizgalska, D., P. Wegrzyn, K. Murzyn, A. Kasza, A. Koj, J. Jura, and B. Jarzab. 2009. Interleukin-1-inducible MCPIP protein has structural and functional properties of RNase and participates in degradation of IL-1beta mRNA. Febs J 276:7386-7399.
24. Kolattukudy, P. E., T. Quach, S. Bergese, S. Breckenridge, J. Hensley, R. Altschuld, G. Gordillo, S. Klenotic, C. Orosz, and J. Parker-Thornburg. 1998. Myocarditis induced by targeted expression of the MCP-1 gene in murine cardiac muscle. Am J Pathol 152:101-111.
25. Baumgarten, G., P. Knuefermann, G. Schuhmacher, V. Vervolgyi, J. von Rappard, U. Dreiner, K. Fink, C. Djoufack, A. Hoeft, C. Grohe, A. A. Knowlton, and R. Meyer. 2006. Toll-like receptor 4, nitric oxide, and myocardial depression in endotoxemia. Shock 25:43-49.
26. Beckman, J. S., and W. H. Koppenol. 1996. Nitric oxide, superoxide, and peroxynitrite: the good, the bad, and ugly. Am J Physiol 271:C1424-1437.
27. Tatsumi, T., K. Akashi, N. Keira, S. Matoba, A. Mano, J. Shiraishi, S. Yamanaka, M. Kobara, N. Hibino, S. Hosokawa, J. Asayama, S. Fushiki, H. Fliss, M. Nakagawa, and H. Matsubara. 2004. Cytokine-induced nitric oxide inhibits mitochondrial energy production and induces myocardial dysfunction in endotoxin-treated rat hearts. J Mol Cell Cardiol 37:775-784.
28. Ferdinandy, P., H. Danial, I. Ambrus, R. A. Rothery, and R. Schulz. 2000. Peroxynitrite is a major contributor to cytokine-induced myocardial contractile failure. Circ Res 87:241-247.
29. Chopra, M., and A. C. Sharma. 2009. Contractile response of norepinephrine is modulated by caspase-3 in adult rat ventricular myocytes isolated from septic rat heart. Pharmacol Res 60:303-313.
30. Lancel, S., O. Joulin, R. Favory, J. F. Goossens, J. Kluza, C. Chopin, P. Formstecher, P. Marchetti, and R. Neviere. 2005. Ventricular myocyte caspases are directly responsible for endotoxin-induced cardiac dysfunction. Circulation 111:2596-2604.
31. Rossi, M. A., M. R. Celes, C. M. Prado, and F. P. Saggioro. 2007. Myocardial structural changes in long-term human severe sepsis/septic shock may be responsible for cardiac dysfunction. Shock 27:10-18.
32. Duriez, P. J., and G. M. Shah. 1997. Cleavage of poly(ADP-ribose) polymerase: a sensitive parameter to study cell death. Biochem Cell Biol 75:337-349.
33. Zanotti-Cavazzoni, S. L., and S. M. Hollenberg. 2009. Cardiac dysfunction in severe sepsis and septic shock. Curr Opin Crit. Care 15:392-397.
34. Alvarez, B., and R. Radi. 2003. Peroxynitrite reactivity with amino acids and proteins. Amino Acids 25:295-311.
35. Mihm, M. J., F. Yu, D. M. Weinstein, P. J. Reiser, and J. A. Bauer. 2002. Intracellular distribution of peroxynitrite during doxorubicin cardiomyopathy: evidence for selective impairment of myofibrillar creatine kinase. Br J Pharmacol 135:581-588.
36. Kumar, A., P. Michael, D. Brabant, A. M. Parissenti, C. V. Ramana, X. Xu, and J. E. Parrillo. 2005. Human serum from patients with septic shock activates transcription factors STAT1, IRF1, and NF-kappaB and induces apoptosis in human cardiac myocytes. J Biol Chem 280:42619-42626.
37. Wesche-Soldato, D. E., R. Z. Swan, C. S. Chung, and A. Ayala. 2007. The apoptotic pathway as a therapeutic target in sepsis. Curr Drug Targets 8:493-500.
38. Fauvel, H., P. Marchetti, C. Chopin, P. Formstecher, and R. Neviere. 2001. Differential effects of caspase inhibitors on endotoxin-induced myocardial dysfunction and heart apoptosis. Am J Physiol Heart Circ Physiol 280:H1608-1614.
39. Hotchkiss, R. S., and D. W. Nicholson. 2006. Apoptosis and caspases regulate death and inflammation in sepsis. Nat Rev Immunol 6:813-822.
40. Dhingra, S., A. K. Sharma, R. C. Arora, J. SlezakJ, and P. K. Singal. 2009. IL-10 attenuates TNF-alpha-induced NF kappaB pathway activation and cardiomyocyte apoptosis. Cardiovasc Res 82:59-66.
41. Chen, D., C. Assad-Kottner, C. Orrego, and G. Torre-Amione. 2008. Cytokines and acute heart failure. Crit. Care Med 36:S9-16.
42. Kumar, A., V. Thota, L. Dee, J. Olson, E. Uretz, and J. E. Parrillo. 1996. Tumor necrosis factor alpha and interleukin 1beta are responsible for in vitro myocardial cell depression induced by human septic shock serum. J Exp Med 183:949-958.
43. Mariappan, N., C. M. Elks, S. Sriramula, A. Guggilam, Z. Liu, O. Borkhsenious, and J. Francis. 2010. NF-kappaB-induced oxidative stress contributes to mitochondrial and cardiac dysfunction in type II diabetes. Cardiovasc Res 85:473-483.
44. Victor, V. M., J. V. Espulgues, A. Hernandez-Mijares, and M. Rocha. 2009. Oxidative stress and mitochondrial dysfunction in sepsis: a potential therapy with mitochondria-targeted antioxidants. Infect Disord Drug Targets 9:376-389.
45. Moss, N. C., W. E. Stansfield, M. S. Willis, R. H. Tang, and C. H. Selzman. (2007). IKKbeta inhibition attenuates myocardial injury and dysfunction following acute ischemia-reperfusion injury. Am J Physiol Heart Circ Physiol 293:H2248-2253.
46. Li, C., R. L. Kao, T. Ha, J. Kelley, I. W. Browder, and D. L. Williams. (2001). Early activation of IKKbeta during in vivo myocardial ischemia. Am J Physiol Heart Circ Physiol 280:H1264-1271.
47. Skaug, B., X. Jiang, and Z. J. Chen. 2009. The role of ubiquitin in NF-kappaB regulatory pathways. Annu Rev Biochem 78:769-796.
48. Carter, R. S., K. N. Pennington, P. Arrate, E. M. Oltz, and D. W. Ballard. 2005. Site-specific monoubiquitination of IkappaB kinase IKKbeta regulates its phosphorylation and persistent activation. J Biol Chem 280:43272-43279.
49. Xia, Z. P., L. Sun, X. Chen, G. Pineda, X. Jiang, A. Adhikari, W. Zeng, and Z. J. Chen. 2009. Direct activation of protein kinases by unanchored polyubiquitin chains. Nature 461:114-119.
50. Niu, J., A. Azfer, and P. E. Kolattukudy. 2008. Protection against lipopolysaccharide-induced myocardial dysfunction in mice by cardiac-specific expression of soluble Fas. J Mol Cell Cardiol 44:160-169.
51. Niu, J., A. Azfer, K. Wang, X. Wang, and P. E. Kolattukudy P E. 2009. Cardiac-targeted expression of soluble fas attenuates doxorubicin-induced cardiotoxicity in mice. J Pharmacol Exp Ther 328:740-748.
52. Azfer, A., J. Niu, L. M. Rogers, F. M. Adamski, and P. E. Kolattukudy. 2006. Activation of endoplasmic reticulum stress response during the development of ischemic heart disease. Am J Physiol Heart Circ Physiol 291:H1411-1420.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory Press, New York (1982) and Sambrook et al., Molecular Cloning: A Laboratory Manual, Cold Spring Harbor Laboratory Press, New York (1989); Methods in Plant Molecular Biology, Maliga et al, Eds., Cold Spring Harbor Laboratory Press, New York (1995); Arabidopsis, Meyerowitz et al, Eds., Cold Spring Harbor Laboratory Press, New York (1994) and the various references cited therein.

Finally, while various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims. The teachings of all patents and other references cited herein are incorporated herein by reference in their entirety to the extent they are not inconsistent with the teachings herein.

TABLE
Heart mass and echocardiography data

| | WT | TG |
|---|---|---|
| n | 6 | 6 |
| Heart/BW, mg/g | 4.5 ± 0.1 | 4.6 ± 0.2 |
| IVSd, mm | 0.58 ± 0.02 | 0.59 ± 0.05 |
| LVIDd, mm | 3.44 ± 0.04 | 3.47 ± 0.04 |
| LVPWd, mm | 0.78 ± 0.04 | 0.75 ± 0.04 |
| IVSs, mm | 1.39 ± 0.04 | 1.42 ± 0.05 |
| LVIDs, mm | 1.69 ± 0.06 | 1.69 ± 0.03 |
| LVPWs, mm | 1.42 ± 0.04 | 1.41 ± 0.05 |
| Ejection fraction, % | 87.4 ± 0.27 | 87.7 ± 0.21 |
| Fractional shortening, % | 50.9 ± 0.31 | 51.9 ± 0.21 |
| LV mass, mg | 65.8 ± 0.11 | 66 ± 0.17 |

What is claimed is:

1. A method of treating a patient in need, said method comprising elevating serum or cellular levels of MCPIP in said patient, wherein said patient in need is exhibiting at least one of the following symptoms associated with sepsis:
systolic blood pressure <90 mm Hg or a reduction of >40 mm Hg from baseline;
decreased left ventricular ejection fraction (LVEF) and an acutely dilated left ventricle, as evidenced by an increased left ventricular end-diastolic volume index (LVEDVI);
low or decreasing cardiac index as calculated by Formula 1.
$$CI = \frac{CO}{BSA} = \frac{SV * HR}{BSA}$$
where
CI=Cardiac index
BSA=Body surface area
SV=Stroke volume
HR=Heart rate
CO=Cardiac output
decreased contractility;
impaired ventricular response to fluid therapy;
lactic acidosis; or
oliguria.

2. The method of claim 1, wherein a low cardiac index is less than 2.6 L/min per square meter.

3. The method of claim 1, wherein a low cardiac index is less than 1.8 L/min/sq. meter.

4. The method of claim 1, wherein elevating MCPIP levels comprises elevating serum levels.

5. The method of claim 1, wherein elevating MCPIP levels comprises elevating intracellular levels.

6. The method of claim 1, wherein elevating intracellular levels comprises elevating MCPIP levels in myocardial cells of said patient in need.

7. The method of claim 1, wherein elevating MCPIP levels comprises directly administering MCPIP to said patient in need.

8. The method of claim 7, wherein directly administering MCPIP to said patient occurs via parenteral administration.

9. The method of claim 7, wherein directly administering MCPIP to said patient occurs via intrabuccal, oral, rectal, pulmonary, ocular, or transdermal administration.

10. The method of claim 7, wherein directly administering MCPIP comprises administering a composition comprising a therapeutically effective amount of MCPIP and a pharmaceutically acceptable excipient.

11. The method of claim 1, wherein elevating MCPIP levels comprises administration of a delivery vehicle that induces expression of MCPIP levels in said patient in need.

12. The method of claim 1, wherein said delivery vehicle is a viral vector comprising an expression construct that comprises a polynucleotide encoding SEQ ID NO. 2, or a variant thereof possessing at least 90 percent identity therewith and which possesses MCPIP activity.

13. The method of claim 1, wherein said delivery vehicle is a cell transfected with an expression construct that comprises a polynucleotide encoding SEQ ID NO. 2, or a variant thereof possessing at least 90 percent identity therewith and which possesses MCPIP activity.

14. The method of claim 1, wherein said delivery vehicle is naked DNA comprising an expression construct that comprises a polynucleotide encoding SEQ ID NO. 2, or a variant thereof possessing at least 90 percent identity therewith and which possesses MCPIP activity.

15. The method of claim 1, wherein said delivery vehicle is a liposome comprising an expression construct that comprises a polynucleotide encoding SEQ ID NO. 2, or a variant thereof possessing at least 90 percent identity therewith and which possesses MCPIP activity.

16. The method of claim 1, wherein elevating MCPIP levels in said patient comprises administering an inducer that upregulates expression of endogenous MCPIP.

17. The method of claim 1, wherein elevating MCPIP levels comprises implanting cells transfected with an expression construct encoding SEQ ID NO. 2 or a variant thereof possessing at least 90 percent identity and possesses MCPIP activity into myocardial cells of said patient.

18. A method of treating a patient experiencing sepsis induced cardiac dysfunction, said method comprising elevating MCPIP levels in said patient.

19. The method of claim 18, wherein said elevating MCPIP levels comprises directly administering MCPIP to said patient.

20. The method of claim 18, wherein said elevating MCPIP levels comprises administering a delivery vehicle comprising an expression construct encoding SEQ ID NO. 2 or a variant thereof possessing at least 90 percent identity and possesses MCPIP activity into myocardial cells of said patient.

21. A method of ameliorating inflammation mediated by NF-κB in a patient in need, said method comprising elevating MCPIP levels in said patient.

22. The method of claim 21, wherein said elevating MCPIP levels comprises directly administering MCPIP to said patient.

23. The method of claim 21, wherein said elevating MCPIP levels comprises administering a delivery vehicle comprising an expression construct encoding SEQ ID NO. 2 or a variant thereof possessing at least 90 percent identity and possesses MCPIP activity into myocardial cells of said patient.

24. The method of claim 21, wherein said patient in need is exhibiting symptoms of arthritis, atherosclerosis, multiple sclerosis, chronic inflammatory demyelinating polyradiculoneuritis, asthma, inflammatory bowel disease, gastritis, systemic inflammatory response syndrome.


SEQUENCE LISTING (4 sequences)

SEQ ID NO: 1 - DNA, 1800 bp, Homo sapiens
  mRNA (1)..(1800)
  ref <308>: DATABASE ACCESSION NUMBER: GenBank/AY920403
  ref <309>: DATABASE ENTRY DATE: 2005-02-05
  ref <313>: RELEVANT RESIDUES IN SEQ ID NO: (1)..(1800)
atgagtggcccctgtggagagaagcctgtcctggaagccagccccaccatgagtctgtgggaatttgaggacagccacagccgtcagggcaccccaaggccgggtcaagagctggccgctgaggaggcctcggccctggaactgcagatgaaggtggacttcttccggaagctgggctattcatccacggagatccacagcgtcctgcagaagctgggcgtccaggcagacaccaacacggtgctgggtgagctggtgaaacacgggacagccaccgagcgggagcgccagacctcaccggacccctgccctcagctccctctagtcccgcggggtggtggcacccctaaggctcccaacctggagcctccactcccagaagaggaaaaggagggcagcgacctgagaccagtggtcatcgatgggagcaacgtggccatgagccatgggaacaaggaggtcttctcctgccggggcatcctgctggcagtgaactggtttctggagcggggccacacagacatcacagtgtttgtgccatcctggaggaaggagcagcctcggcccgacgtgcccatcacagaccagcacatcctgcgggaactggagaagaagaagatcctggtgttcacaccatcacgacgcgtgggtggcaagcgggtggtgtgctatgacgacagattcattgtgaagctggcctacgagtctgacgggatcgtggtttccaacgacacataccgtgacctccaaggcgagcggcaggagtggaagcgcttcatcgaggagcggctgctcatgtactccttcgtcaatgacaagtttatgccccctgatgacccactgggccggcacgggcccagcctggacaacttcctgcgtaagaagccactcactttggagcacaggaagcagccgtgtccctatggaaggaaatgcacctatgggatcaagtgccgattcttccacccagagcggccaagctgcccccagcgctctgtggcagatgagctccgtgccaatgctctcctctcaccccccagagccccaagcaaggacaaaaatggccggcggccttcaccttcatcccagtccagctctctgctaacagagagtgagcagtgcagcctggatgggaagaagctgggggcccaggcatccccagggtcccgccaagagggtctaacacagacctatgccccatcaggcaggagcctcgcacctagcgggggcagtggcagcagctttgggcccacagactggctcccacagacgctggactcactcccgtacgtctcccaggattgcctggactcgggcattggctccctggagagccagatgtcggaactttggggggttcgaggaggaggccctggtgagccgggcccaccccgagccccttacacgggctacagtccctatggatctgagctcccagccaccgcagccttctctgcctttggccgggccatgggtgctggccacttcagtgtccctgccgactacccacccgcgccccctgcctttccacctcgagagtactggtctgaaccatacccactgcccccacccacatcagtccttcaggagcccccagtgcagagcccaggggctggcaggagcccgtggggcagggcaggcagcctggccaaggagcaggccagcgtgtatactaagctgtgtggtgtgtttcccccgcacctggtggaggctgtgatggggcgcttcccacagctcctggacccccagcagctggctgccgagatcctctcctacaagtcccagcaccccagtgagtaa

SEQ ID NO: 2 - protein, 599 aa, Homo sapiens
  PEPTIDE (1)..(599)
  ref <308>: DATABASE ACCESSION NUMBER: GenBank/AY920403
  ref <309>: DATABASE ENTRY DATE: 2005-02-05
  ref <313>: RELEVANT RESIDUES IN SEQ ID NO: (1)..(599)
MSGPCGEKPVLEASPTMSLWEFEDSHSRQGTPRPGQELAAEEASALELQMKVDFFRKLGYSSTEIHSVLQKLGVQADTNTVLGELVKHGTATERERQTSPDPCPQLPLVPRGGGTPKAPNLEPPLPEEEKEGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAYESDGIVVSNDTYRDLQGERQEWKRFIEERLLMYSFVNDKFMPPDDPLGRHGPSLDNFLRKKPLTLEHRKQPCPYGRKCTYGIKCRFFHPERPSCPQRSVADELRANALLSPPRAPSKDKNGRRPSPSSQSSSLLTESEQCSLDGKKLGAQASPGSRQEGLTQTYAPSGRSLAPSGGSGSSFGPTDWLPQTLDSLPYVSQDCLDSGIGSLESQMSELWGVRGGGPGEPGPPRAPYTGYSPYGSELPATAAFSAFGRAMGAGHFSVPADYPPAPPAFPPREYWSEPYPLPPPTSVLQEPPVQSPGAGRSPWGRAGSLAKEQASVYTKLCGVFPPHLVEAVMGRFPQLLDPQQLAAEILSYKSQHPSE

SEQ ID NO: 3 - DNA, 1791 bp, Mus musculus
  mRNA (1)..(1791)
  ref <308>: DATABASE ACCESSION NUMBER: GenBank/AY920404
  ref <309>: DATABASE ENTRY DATE: 2005-02-05
  ref <313>: RELEVANT RESIDUES IN SEQ ID NO: (1)..(1791)
atgagtgacccttgtggaacgaagcctgtccaagaatccaaccccaccatgagtctgtggagtcttgaggacagacacagcagccagggtcgacctcagccagaccaggatcctgtggctaaagaggcccctacttccgagctgcagatgaaggtggactttttccgtaaactggggtactcgtcctctgagatccacagtgtcctgcagaagctgggagtccaagcagacaccaacacggtgctaggggaattggtgaagcatggctcagctactgaacgagaatgccaggccctgacggcccccagcccccagccccctctggtgccccggggtggaagcacccccaagccttccactctagaaccctcactcccagaggaggacagagagggcagcgacctgagacctgtggtcatcgacggaagcaatgtggccatgagccatgggaacaaggaagtcttctcttgccggggcattctgctggctgtgaactggtttctggagcgaggccacacagatattaccgtgtttgtgccatcttggaggaaggaacagcctcgaccagatgtgcctatcacagaccagcacatccttcgggaactagagaaaaagaagatcttggtgttcacgccatccaggcgggtcggcggcaagcgcgtggtgtgctatgatgaccgcttcattgtgaagctggccttcgaatccgacggagtggtggtctccaatgacacgtaccgagacctccaaggcgagaggcaggagtggaaacgcttcatcgaggagcggctgctcatgtactccttcgtcaatgacaagttcatgccccctgacgaccctttaggacggcatgggcctagcctggacaacttccttcgtaagaaaccactgccttctgagcacaggaagcagccatgcccctatgggaagaaatgtacgtatgaaatcaagtgccgatttttccaccctgagcggccaagccgtccccagcgctctgtggccgatgagctccgtgccaacgctctcctctcacctcccaggactccagtcaaggacaaaagtagccagaggccttcccctgcctctcagtccagctctgtgtccctagaggctgaaccaggcagcctggatgggaaaaagctgggtgccaggtcatctccgggtccccaccgagaaggctcaccgcagacctgtgctccagctggcaggagcctccctgttagtgggggcagctttgggcccacagagtggcttgcacacacccaggactcactcccatacacctcccaggagtgccttgattcaggcattggttccctggagagccagatgtcagaattatggggcgtgcgaggaggcagccctggggagtcgggccccactcggggcccctatgcaggttatcacagctatggatccaaggtcccagcagcaccttccttttctccttttagaccagccatgggtgctggccacttcagtgtccccaccgactatgtgcccccgccacccacctacccatccagagagtactggtctgagccgtacccattacccccacccactcctgtccttcaggagccccagagacccagccccggggctggtgggggcccctggggcagggtgggtgacctggccaaagaaagggctggtgtatataccaagctgtgtggtgtcttccccccacacctggtagaagctgtaatgagacgcttcccacagctgctggatccgcagcagctggccgcagagatcctgtcttacaagtcccagcacctcagtgagtaa

SEQ ID NO: 4 - protein, 596 aa, Mus musculus
  PEPTIDE (1)..(596)
  ref <308>: DATABASE ACCESSION NUMBER: GenBank
  ref <309>: DATABASE ENTRY DATE: 2005-02-05
  ref <313>: RELEVANT RESIDUES IN SEQ ID NO: (1)..(596)
MSDPCGTKPVQESNPTMSLWSLEDRHSSQGRPQPDQDPVAKEAPTSELQMKVDFFRKLGYSSSEIHSVLQKLGVQADTNTVLGELVKHGSATERECQALTAPSPQPPLVPRGGSTPKPSTLEPSLPEEDREGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAFESDGVVVSNDTYRDLQGERQEWKRFIEERLLMYSFVNDKFMPPDDPLGRHGPSLDNFLRKKPLPSEHRKQPCPYGKKCTYEIKCRFFHPERPSRPQRSVADELRANALLSPPRTPVKDKSSQRPSPASQSSSVSLEAEPGSLDGKKLGARSSPGPHREGSPQTCAPAGRSLPVSGGSFGPTEWLAHTQDSLPYTSQECLDSGIGSLESQMSELWGVRGGSPGESGPTRGPYAGYHSYGSKVPAAPSFSPFRPAMGAGHFSVPTDYVPPPPTYPSREYWSEPYPLPPPTPVLQEPQRPSPGAGGGPWGRVGDLAKERAGVYTKLCGVFPPHLVEAVMRRFPQLLDPQQLAAEILSYKSQHLSE